(19) United States
(12) Patent Application Publication
(10) Pub. No.: US 2009/0197794 A1
(43) Pub. Date: Aug. 6, 2009

(54) Methods of Diagnosing and Treating an Inflammatory Response

(76) Inventors: William C. Aird, Brookline, MA (US); Kiichiro Yano, Charlestown, MA (US)

Correspondence Address: CLARK & ELBING LLP, 101 FEDERAL STREET, BOSTON, MA, 02110, US

(21) Appl. No.: 11/988,579

(22) PCT Filed: Jul. 13, 2006
(86) PCT No.: PCT/US06/27402

§ 371 Date: Jan. 22, 2009

Related U.S. Application Data
(60) Provisional application No. 60/698,997, filed on Jul. 13, 2005.

(51) Int. Cl. A61K 38/12; C12Q 1/00; C12Q 1/68; G01N 33/53; C40B 30/00; A61K 38/28; A61K 31/56

(52) U.S. Cl. 514/4; 435/4; 435/6; 435/7.1; 506/7; 514/12; 514/171

(57) ABSTRACT

The present invention relates to the discovery that VEGF, PlGF, and sFlt-1 levels are increased in inflammatory response such as in sepsis, severe sepsis, or septic shock. Additionally, the invention provides methods of identifying treatments as well as providing treatments for such an inflammatory response, which include decreasing VEGF or PlGF levels, or increasing sFlt-1 or PlGF levels.

### STATEMENT AS TO FEDERALLY FUNDED RESEARCH

The present research was supported by a grant from the National Heart, Lung, and Blood Institute of the National Institutes of Health (Number PO1 HL076540). The U.S. Government may therefore have certain rights to this invention.

### BACKGROUND OF THE INVENTION

The invention relates to fields of diagnosing and treating an inflammatory response.

Inflammatory response is associated with life-threatening conditions such as sepsis, severe sepsis, and septic shock. Sepsis is defined as the systemic inflammatory response to infection. Severe sepsis is associated with organ dysfunction, is common (750,000 new cases each year in the USA), and has a high mortality rate (30%). The incidence is predicted to increase by 1.5% per year, owing to aging of the population, and the wider use of immunosuppressive agents and invasive procedures. Host response to infection is complex and involves an elaborate array of soluble mediators (e.g., components of the inflammatory and coagulation cascades) and cells (e.g., platelets, monocytes, and endothelial cells). Previous efforts to block one or another component of the inflammatory or coagulation pathways have had little impact on survival. Of the many agents and drugs that have been tested, only two have demonstrated efficacy in phase 3 clinical trials: murine monoclonal antibody to human tumor necrosis factor (TNF)-α and human recombinant activated protein C (rhAPC). However, despite these interventions, mortality rates remain high at 25-30%. Clearly, advances in therapy will be contingent upon an improved understanding of sepsis pathophysiology. Given the high mortality of untreated severe sepsis and, prior to the present invention, a lack of effective treatments, there is a need for better diagnostic and treatment tools for inflammatory responses such as sepsis.

Vascular endothelial growth factor (VEGF)/vascular permeability factor (VPF) was first identified and characterized by Dvorak and colleagues as a potent stimulator of endothelial permeability. VEGF was subsequently reported to promote proliferation, migration and survival of endothelial cells. VEGF (also termed VEGF-A) is a member of a growing family of related proteins that include VEGF-B, -C, -D and placenta derived growth factor (PlGF). VEGF binds to two transmembrane receptors, namely Flt-1 and Flk-1, whereas PlGF binds to Flt-1 alone. Within the vessel wall, Flk-1 is selectively expressed in endothelium. Flt-1 is present on both endothelial cells and monocytes.

### SUMMARY OF THE INVENTION

In a first aspect, the invention provides a method of diagnosing an inflammatory response (e.g., severe sepsis or septic shock) in a test subject (e.g., a human), the method including analyzing the level of sFlt-1 expression or activity in a sample isolated from the test subject, where an increased level of sFlt-1 expression or activity in the sample relative to the level found in an unaffected subject indicates that the test subject has the inflammatory response. The method may further include analyzing the level of at least one of VEGF, PlGF, TNF-α, IL-6, D-dimer, E-selectin, P-selectin, ICAM-1, VCAM-1, Cox-2, or PAI-1.

The invention also provides a method of identifying a candidate compound useful for treating a subject with an inflammatory response, the method including contacting sFlt-1 with a compound (e.g., a compound selected from a chemical library); and measuring the activity of the sFlt-1, where an increase in sFlt-1 activity in the presence of the compound relative to sFlt-1 activity in the absence of the compound identifies the compound as a candidate compound for treating a subject with an inflammatory response. The measuring step may include measuring binding of at least one of VEGF or PlGF to sFlt-1.

The invention also provides a method of identifying a candidate compound useful for treating a subject with an inflammatory response, the method including contacting a cell (e.g., a cell in a mammal) or cell extract including a polynucleotide encoding sFlt-1 with a compound (e.g., a compound selected from a chemical library); and measuring the level of sFlt-1 expression in the cell or cell extract, where an increased level of sFlt-1 expression in the presence of the compound relative to the level in the absence of the compound identifies the compound as a candidate compound for treating a subject with an inflammatory response. The method may further include administering to the mammal lipopolysaccharide prior to the contacting step.

The invention also provides a method of treating a subject (e.g., a human) with an inflammatory response (e.g., severe sepsis or septic shock), the method including administering to the subject a therapeutically effective amount of a composition (e.g., a composition including sFlt-1) that increases sFlt-1 expression or activity. The method may further include administering a treatment selected from the group consisting of antimicrobials, fluids, vasopressors, corticosteroids, activated protein C, glucose with insulin, mechanical ventilation, renal replacement therapy, and sedation.

The invention also provides a method of diagnosing an inflammatory response (e.g., severe sepsis or septic shock) in a test subject (e.g., a human), the method including analyzing the level of PlGF expression or activity in a sample isolated from the test subject, where an alteration (e.g., an increase or a decrease) in the level of PlGF expression or activity in the sample relative to the level in an unaffected subject indicates that the test subject has the inflammatory response. The method may further include analyzing the level of at least one of VEGF, PlGF, TNF-α, IL-6, D-dimer, E-selectin, P-selectin, ICAM-1, VCAM-1, Cox-2, or PAI-1.

The invention also provides a method of identifying a candidate compound useful for treating a subject with an inflammatory response, the method including contacting PlGF with a compound (e.g., a compound selected from a chemical library); and measuring the activity of the PlGF, where an alteration (e.g., an increase or a decrease) in PlGF activity in the presence of the compound relative to PlGF activity in the absence of the compound identifies the compound as a candidate compound for treating a subject with an inflammatory response.

The invention also provides a method of identifying a candidate compound useful for treating a subject with an inflammatory response, the method including contacting a cell (e.g., a cell in a mammal) or cell extract including a polynucleotide encoding PlGF with a compound (e.g., a compound selected from a chemical library); and measuring the level of PlGF expression in the cell or cell extract, where an alteration (e.g., an increase or a decrease) in the level of PlGF expression in the presence of the compound relative to the level in the absence of the compound identifies the compound as a candidate compound for treating a subject with an inflammatory response. The method may further include administering to the mammal lipopolysaccharide prior to the contacting step.

The invention also provides a method of identifying a candidate compound for treating a subject (e.g., a human) with an inflammatory response (e.g., severe sepsis or septic shock), the method including contacting a PlGF receptor (e.g., neuropilin-1 or VEGFR-1, or a fragment thereof), or a PlGF-binding fragment thereof, with a compound (e.g., a compound selected from a chemical library); and measuring the binding of the compound to the receptor, where specific binding of the compound to the PlGF receptor or the fragment thereof indicates the compound is a candidate compound for treating a subject with an inflammatory response.

The invention also provides a method of treating a subject (e.g., a human) with an inflammatory response (e.g., severe sepsis or septic shock), the method including administering to the subject a therapeutically effective amount of a composition (e.g., a composition including PlGF, a nucleic acid that encodes PlGF, or a fragment thereof with PlGF activity, antibody specifically binds PlGF, or a PlGF-binding fragment thereof, an RNA that interferes with the mRNA coding for the PlGF protein) that alters (e.g., increases or decreases) the expression or activity of PlGF. The may further include administering a treatment selected from the group consisting of antimicrobials, fluids, vasopressors, corticosteroids, activated protein C, glucose with insulin, mechanical ventilation, renal replacement therapy, and sedation.

The invention also provides a method of treating a subject (e.g., a human) with an inflammatory response (e.g., severe sepsis or septic shock) which includes administering to the subject a therapeutically effective amount of a composition that alters (e.g., increases or decreases) the expression or activity of a PlGF receptor (e.g., neuropilin-1 or VEGFR-1). The method may further include administering a treatment selected from the group consisting of antimicrobials, fluids, vasopressors, corticosteroids, activated protein C, glucose with insulin, mechanical ventilation, renal replacement therapy, and sedation.

The invention also provides a method of diagnosing an inflammatory response (e.g., severe sepsis or septic shock) in a test subject (e.g., a human), the method including analyzing the level of VEGF expression or activity in a sample isolated from the test subject, where an increased level of VEGF expression or activity in the sample relative to the level found in an unaffected subject indicates that the test subject has the inflammatory response. The method may further include analyzing the level of at least one of PlGF, sFlt-1, TNF-α, IL-6, D-dimer, E-selectin, P-selectin, ICAM-1, VCAM-1, Cox-2, or PAI-1.

The invention also provides a method of identifying a candidate compound useful for treating a subject with an inflammatory response, the method including contacting VEGF with a compound (e.g., a compound selected from a chemical library); and measuring the activity of the VEGF, where a decrease in VEGF activity in the presence of the compound relative to VEGF activity in the absence of the compound identifies the compound as a candidate compound for treating a subject with an inflammatory response.

The invention also provides a method of identifying a candidate compound useful for treating a subject with an inflammatory response, the method including contacting a cell (e.g., a cell in a mammal) or cell extract including a polynucleotide encoding VEGF with a compound (e.g., a compound selected from a chemical library); and measuring the level of VEGF expression in the cell or cell extract, where a decreased level of VEGF expression in the presence of the compound relative to the level in the absence of the compound identifies the compound as a candidate compound for treating a subject with an inflammatory response. The method may further include administering to the mammal lipopolysaccharide prior to the contacting step.

The invention also provides a method of identifying a candidate compound for treating a subject with an inflammatory response, the method including contacting a VEGF receptor (e.g., neuropilin-1, VEGFR-1, VEGFR-2, or a fragment thereof), or a VEGF binding fragment thereof, with a compound (e.g., a compound selected from a chemical library); and measuring the binding of the compound to the receptor, where specific binding of the compound to the VEGF receptor or the fragment thereof indicates the compound is a candidate compound for treating a subject with an inflammatory response.

The invention also provides a method of treating a subject (e.g., a human) with an inflammatory response (e.g., severe sepsis or septic shock), the method including administering to the subject a therapeutically effective amount of a composition (e.g., a composition including an antibody specifically binds VEGF, or a VEGF-binding fragment thereof that decreases the expression or activity of VEGF, or a composition including RNA that interferes with the mRNA coding for VEGF). The method may further include administering a treatment selected from the group consisting of antimicrobials, fluids, vasopressors, corticosteroids, activated protein C, glucose with insulin, mechanical ventilation, renal replacement therapy, and sedation.

The invention also provides a method of treating a subject (e.g., a human) with an inflammatory response (e.g., severe sepsis or septic shock), the method including administering to the subject a therapeutically effective amount of a composition that decreases the expression or activity of a VEGF receptor (e.g., neuropilin-1, VEGFR-1, or VEGFR-2). The method may further include administering a treatment selected from the group consisting of antimicrobials, fluids, vasopressors, corticosteroids, activated protein C, glucose with insulin, mechanical ventilation, renal replacement therapy, and sedation.

By “inflammatory response” is meant the activation of the immune system in a subject, for instance, a mammal such as a human. An inflammatory response may involve the induction of cytokines, VEGF, PlGF, and sFlt-1 and may result, for example, from an autoimmune disease, from contact of the mammal with a virus, a gram-negative bacterium, a gram-positive bacterium, or a component thereof, such as lipopolysaccharide.

By “vascular endothelial growth factor (VEGF)” is meant a mammalian growth factor that is homologous to the growth factor defined in U.S. Pat. Nos. 5,332,671; 5,240,848; 5,194,596; and Charnock-Jones et al. (Biol. Reproduction, 48:1120-1128, 1993), and has VEGF biological activity. VEGF exists as a glycosylated homodimer and includes at least four different alternatively spliced isoforms. The biological activity of native VEGF includes the promotion of selective growth of vascular endothelial cells or umbilical vein endothelial cells and induction of angiogenesis. As used herein, VEGF includes any VEGF family member or isoform (e.g., VEGF-A, VEGF-B, VEGF-C, VEGF-D, VEGF-E, VEGF189, VEGF165, or VEGF 121). VEGF may be the VEGF 121 or VEGF165 isoform (Tischer et al., J Biol Chem 266:11947-11954, 1991; Neufed et al. Cancer Metastasis 15:153-158, 1996), which is described in U.S. Pat. Nos. 6,447,768; 5,219,739; and 5,194,596, hereby incorporated by reference. Also included are mutant forms of VEGF such as the KDR-selective VEGF and Flt-selective VEGF described in Gille et al. (J Biol Chem 276:3222-3230, 2001). Although human VEGF is preferred, the invention is not limited to human forms and can include other animal forms of VEGF (e.g., mouse, rat, dog, or chicken).

By “placental growth factor (PlGF)” is meant a mammalian growth factor that is homologous to the protein defined by GenBank accession number P49763 and that has PlGF biological activity. PlGF is a glycosylated homodimer belonging to the VEGF family and can be found in two distinct isoforms through alternative splicing mechanisms. PlGF is expressed by cyto- and syncytiotrophoblasts in the placenta and PlGF biological activities include induction of proliferation, migration, and activation of endothelial cells, particularly trophoblast cells.

By “soluble Flt-1 (sFlt-1)” (also known as sVEGF-R1) is meant the soluble form of the Flt-1 receptor, that is homologous to the protein defined by GenBank accession number U01134, and that has sFlt-1 biological activity. The biological activity of an sFlt-1 polypeptide may be assayed using any standard method, for example, by assaying sFlt-1 binding to VEGF. sFlt-1 lacks the transmembrane domain and the cytoplasmic tyrosine kinase domain of the Flt-1 receptor. sFlt-1 can bind to VEGF and PlGF bind with high affinity, but it cannot induce proliferation or angiogenesis and is therefore functionally different from the Flt-1 and KDR receptors. sFlt-1 was initially purified from human umbilical endothelial cells and later shown to be produced by trophoblast cells in vivo. As used herein, sFlt-1 includes any sFlt-1 family member or isoform.

By “alteration” is meant a change (increase or decrease) in the expression levels of a gene or polypeptide as detected by standard art known methods such as those described herein. As used herein, an increase or decrease includes at least 10% change in expression levels, such as a 25% change, a 40% change, or a 50% or greater change in expression levels. “Alteration” can also indicate a change (increase or decrease) in the biological activity of any of the polypeptides of the invention (e.g., sFlt-1, VEGF, or PlGF). Examples of biological activity for PlGF or VEGF include binding to receptors as measured by immunoassays, ligand binding assays or Scatchard plot analysis, and induction of cell proliferation or migration as measured by BrdU labeling, cell counting experiments, or quantitative assays for DNA synthesis such as ³H-thymidine incorporation. Examples of biological activity for sFlt-1 include binding to PlGF and VEGF as measured by immunoassays, ligand binding assays, or Scatchard plot analysis. Additional examples of biological activity for each of the polypeptides are described herein. As used herein, an increase or decrease includes a 10% change in biological activity, a 25% change, a 40% change, or a 50% or greater change in biological activity.

By “compound” is meant any small molecule chemical compound, antibody, nucleic acid molecule, or polypeptide, or fragments thereof.

By “fragment” is meant a portion of a polypeptide or nucleic acid molecule. This portion contains, e.g., at least 10%, 20%, 30%, 40%, 50%, or 60% of the entire length of the reference nucleic acid molecule or polypeptide. A fragment may contain at least 5, 10, 20, 30, 40, 50, 60, 70, 80, 90, 100, 200, 300, 400, 500, 600, 700, 800, 900, or 1000 nucleotides or amino acids.

By “homologous” is meant any gene or protein sequence that bears at least 30%, 40%, 50%, 60%, 70%, 80%, 90%, 95%, or 99% or more homology to a known gene or protein sequence over the length of the comparison sequence. A “homologous” protein can also have at least one biological activity of the comparison protein. For polypeptides, the length of comparison sequences will generally be at least 16, 20, 25, or 35 amino acids. For nucleic acids, the length of comparison sequences will generally be at least 50, 60, 75, or 110 nucleotides. “Homology” can also refer to a substantial similarity between an epitope used to generate antibodies and the protein or fragment thereof to which the antibodies are directed. In this case, homology refers to a similarity sufficient to elicit the production of antibodies that can specifically recognize the protein at issue.

By “chimeric antibody” is meant a polypeptide comprising at least the antigen-binding portion of an antibody molecule linked to at least part of another protein (typically an immunoglobulin constant domain).

By “humanized antibody” is meant an immunoglobulin amino acid sequence variant or fragment thereof that is capable of binding to a predetermined antigen. Ordinarily, the antibody will contain both the light chain as well as at least the variable domain of a heavy chain. The antibody also may include the CH1, hinge, CH2, CH3, or CH4 regions of the heavy chain. The humanized antibody comprises a framework region (FR) having substantially the amino acid sequence of a human immunoglobulin and a complementarity determining region (CDR) having substantially the amino acid sequence of a non-human immunoglobulin (the “import” sequences).

Generally, a humanized antibody has one or more amino acid residues introduced into it from a source that is non-human. In general, the humanized antibody will comprise substantially all of at least one, and typically two, variable domains (Fab, Fab′, F(ab′)₂, Fabc, Fv) in which all or substantially all of the CDR regions correspond to those of a non-human immunoglobulin and all or substantially all of the FR regions are those of a human immunoglobulin consensus sequence. The humanized antibody optimally will comprise at least a portion of an immunoglobulin constant region (Fc), typically that of a human immunoglobulin.

By “hybridize” is meant pair to form a double-stranded molecule between complementary polynucleotide sequences, or portions thereof, under various conditions of stringency. (See, e.g., Wahl and Berger (1987) Methods Enzymol. 152:399; Kimmel, Methods Enzymol 152:507, 1987.) For example, stringent salt concentration will ordinarily be less than about 750 mM NaCl and 75 mM trisodium citrate, e.g., less than about 500 mM NaCl and 50 mM trisodium citrate, or less than about 250 mM NaCl and 25 mM trisodium citrate. Low stringency hybridization can be obtained in the absence of organic solvent, e.g., formamide, while high stringency hybridization can be obtained in the presence of at least about 35% or at least 50% formamide. Stringent temperature conditions will ordinarily include temperatures of at least about 30° C., e.g., at least about 37° C. or at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In one embodiment, hybridization will occur at 30° C. in 750 mM NaCl, 75 mM trisodium citrate, and 1% SDS. In another embodiment, hybridization will occur at 37° C. in 500 nM NaCl, 50 mM trisodium citrate, 1% SDS, 35% formamide, and 100 μg/ml denatured salmon sperm DNA (ssDNA). In yet another embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps may be less than about 30 mM NaCl and 3 mM trisodium citrate, and may be less than about 15 mM NaCl and 1.5 mM trisodium citrate. Stringent temperature conditions for the wash steps will ordinarily include a temperature of at least about 25° C., 42° C., or 68° C. In one embodiment, wash steps will occur at 25° C. in 30 mM NaCl, 3 mM trisodium citrate, and 0.1% SDS. In another embodiment, wash steps will occur at 42° C. in 15 mM NaCl, 1.5 mM trisodium citrate, and 0.1% SDS. In yet another embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc Natl Acad Sci USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

By “specifically binds” is meant a compound or antibody which recognizes and binds a polypeptide of the invention but that does not substantially recognize and bind other molecules in a sample, for example, a biological sample, which naturally includes a polypeptide of the invention. In one example, an antibody that specifically binds sFlt-1 does not bind Flt-1.

By “subject” is meant a mammal, including, but not limited to, a human or non-human mammal, such as a bovine, equine, canine, ovine, or feline.

By “biological sample” or “sample” is meant a sample obtained from an organism or from components (e.g., cells) of an organism. The sample may be of any biological tissue or fluid. Frequently the sample will be a “clinical sample” which is a sample derived from a subject. Such samples include, but are not limited to, sputum, blood, blood cells (e.g., white cells), tissue or fine needle biopsy samples, urine, peritoneal fluid, and pleural fluid, or cells. Biological samples may also include sections of tissues such as frozen sections taken for histological purposes.

By “substantially identical” is meant an amino acid sequence which differs only by conservative amino acid substitutions, for example, substitution of one amino acid for another of the same class (e.g., valine for glycine, arginine for lysine, etc.) or by one or more non-conservative substitutions, deletions, or insertions located at positions of the amino acid sequence which do not destroy the function of the protein. The amino acid sequence may be at least 70%, 80%, 90%, 95%, 98%, or 99% homologous to another amino acid sequence. Methods to determine identity are available in publicly available computer programs. Computer program methods to determine identity between two sequences include, but are not limited to, the GCG program package (Devereux et al., Nucleic Acids Research 12: 387, 1984), BLASTP, BLASTN, and FASTA (Altschul et al., J. Mol. Biol. 215:403 (1990). The well-known Smith Waterman algorithm may also be used to determine identity. The BLAST program is publicly available from NCBI and other sources (BLAST Manual, Altschul, et al., NCBI NLM NIH, Bethesda, Md. 20894; BLAST 2.0 at http://www.ncbi.nlm.nih.gov/blast/). These software programs match similar sequences by assigning degrees of homology to various substitutions, deletions, and other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine.

By “treating” is meant administering or prescribing a pharmaceutical composition for the cure, stabilization, amelioration, or prevention of a disease, condition, or response (e.g., an inflammatory response). This term includes active treatment, that is, treatment directed specifically toward improvement, and also includes causal treatment, that is, treatment directed toward removal of the cause of the disease, condition, or response. In addition, this term includes palliative treatment, that is, treatment designed for the relief of symptoms rather than the curing; preventive treatment, that is, treatment directed to prevention; and supportive treatment, that is, treatment employed to supplement another specific therapy directed toward the improvement. The term “treating” also includes symptomatic treatment, that is, treatment directed toward constitutional symptoms of the disease, condition or response.

By “an effective amount” is meant an amount of a compound or combination of compounds capable of treating at least one symptom of a disease or condition. The therapeutically effective amount of an active compound or combination may vary depending upon the manner of administration, the age, body weight, and general health of the subject.

Other features and advantages of the invention will be apparent from the following Detailed Description, the drawings, and the claims.

### BRIEF DESCRIPTION OF THE DRAWINGS

FIG. 1 is a schematic diagram of the VEGF family of proteins and corresponding receptors. VEGF interacts with sVEGFR-1 (sFlt-1), VEGFR-1 (Flt-1), VEGFR-2 (Flk-1/KDR), and neuropilin-1; PlGF interacts with sVEGFR-1 (sFlt-1), VEGFR-1 (Flt-1), and neuropilin-1.

FIG. 2 is a schematic diagram of leukocyte attachment and migration outside of vesicles during an inflammatory response. This process involves binding of the leukocyte to ICAM-1 and VCAM-1 present on the endothelial cells of the blood vessel.

FIG. 3 is a drawing illustrating NF-κB induced activation of endothelial adhesion molecules and chemokines.

FIGS. 4a and 4b are a set of graphs showing IL-6 (plasma), TNF-α(serum), PlGF (plasma), and VEGF (plasma) levels circulating in control mice, and mice either treated with LPS or cecal ligation and puncture mice, at 6 hours, 12 hours, and 24 hours. Increases over control mice in VEGF and PlGF are observed for both mouse models.

FIG. 4c is a set of graphs showing PlGF and VEGF levels in mouse pneumonia model at 6 and 24 h. FIG. 4d is a set of graphs showing PlGF and VEGF levels in human endotoxemia model at time points indicated. Subjects were administered LPS or saline (placebo). FIG. 4e is a set of graphs showing plasma PlGF and VEGF levels in randomly chosen patients with severe sepsis plotted against time in the intensive care unit (ICU). Each line represents an individual patient (P).

FIG. 5 is a set of images and graphs showing a pronounced induction of vascular leakage in lung, kidney and liver following lung infection.

FIG. 6 is a set of graphs showing a pronounced increase in plasma PlGF and IL6 levels, but not VEGF levels following lung infection.

FIG. 7 is a schematic diagram of an experimental procedure used to determine the effect of sFlt-1 on the response to LPS injection. Briefly mice are infected with an adenoviral vector containing either a gene encoding sFlt-1 or a control gene. Four days following infection, the mice receive an interperitoneal injection of 18 mg/kg LPS. Five days following infection, physiological evaluation, including echocardiogram and electrocardiogram (ECG) and evaluation of samples from the mice including blood (e.g., serum and plasma) and organs (e.g., heart, liver, spleen, lungs, kidney, brain) are evaluated. Analyses of RNA levels, protein levels, and histology are performed.

FIGS. 8a-8c are graphs demonstrating that complete blockage of free VEGF and PlGF is achieved in the mice infected with the gene coding sFlt-1 as compared to control mice.

FIGS. 9a and 9b are images and a graph showing the effect of sFlt-1 overexpression on vascular permeability in mouse model of endotoxemia. Mice were injected with adenovirus overexpressing GFP (CTRL-ad) or sFlt-1 (sFlt-ad). Three days later, the animals were administered saline (control) or LPS IP. Twenty-four hours later, the animals were injected intravenously with 0.1 ml of 1% Evans blue dye. After 40 min, the mice were perfused, and the brain (Br), lung (Lu), heart (H), liver (Li), kidney (Ki), and spleen (Sp) were harvested and incubated in formamide for 3 days to elute Evans blue dye. FIG. 9a shows whole mount photomicrographs of organs. In each group the left-most specimen is from control untreated mice, the middle specimen from CTRL-ad-treated endotoxemic mice, and the right specimen from sFlt-1-ad-treated endotoxemic mice (in the case of the kidney, two specimens are shown for each condition). FIG. 9b shows quantitation of Evans blue extravasation (OD at 620 nm), *; p<0.05, **; p<0.01, ***; p<0.0001.

FIG. 10a is a graph showing increased survival of mice infected with an adenoviral vector containing the sFlt-1 gene followed by LPS administration (n=24) as compared to control mice infected with a control vector followed by LPS administration (n=24). Twenty two of the 24 mice expressing sFlt-1 survived 96 hours following LPS treatment, as compared to six of the 24 mice expressing a control gene.

FIGS. 10b-10e are graphs showing survival studies in mouse models of sepsis. FIG. 10b shows survival curves for mice overexpressing GFP (CTRL-as), sFlt-1 (sFlt-ad), PlGF (PlGF-ad), and VEGF (VEGF-ad) and injected IP with LPS. Circulating VEGF, PlGF and sFlt-1 at 24 hr after LPS injection was VEGF: 5.08+1.41 ng/ml, PlGF: 28.23+5.84 pg/ml and 20.64+5.20 ng/ml respectively. FIG. 10c shows survival curves for mice overexpressing GFP (CTRL-as) or sFlt-1 (sFltad) and subjected to CLP. FIG. 10d shows survival curves for endotoxemic mice pre-treated with IP injection of antibodies against Flk-1. FIG. 10e shows survival curves for endotoxemic mice pretreated with IP injection of antibodies against Flt-1.

FIGS. 10f and 10g are graphs showing a therapeutic effect of soluble sFlt-1 peptide on sepsis mortality. Survival curves for mice intravenously injected with sFlt-1 peptide or same volume of PBS (control) 1 h following LPS injection (FIG. 10f) or CLP (FIG. 10g).

FIGS. 10h and 10i are graphs showing survival studies of PlGF (−/−) mice and mice treated with an anti-PlGF antibody. Decreased amounts of PlGF or PlGF activity are associated with an increased mortality rate.

FIGS. 11a and 11b are graphs showing that levels of IL-6 and D-dimer are increased in control mice treated with LPS over mice (both control and sFlt-1 expressing) not treated with LPS. Similar levels of both IL-6 and D-dimer are observed in LPS-untreated mice as in mice infected with an adenoviral vector containing a gene coding for sFlt-1 and treated with LPS.

FIGS. 12a-12o are images and graphs showing the effect of sFlt-1 overexpression on cardiac function in mouse model of endotoxemia. Mice infected with an adenoviral vector containing a gene coding for sFlt-1 followed by LPS treated have cardiac function similar to mice not treated with LPS, whereas control mice treated with LPS exhibit altered cardiac activity. Mice were injected with adenovirus overexpressing GFP (CTRL-ad, C) or sFlt-1 (sFlt-ad, S) (FIGS. 12a, 12h, 12i, 12l, and 12m). Three days later, the animals were administered saline (control) or LPS IP, or subjected to CLP. Alternatively, mice were injected IV with PBS (C) or sFlt-1 peptide (S) one hour following LPS injection or CLP procedure (FIGS. 12j, 12k, 12n, and 12o). FIGS. 12a-12e show results as measured by echocardiogram, and FIGS. 12f and 12g show electrocardiogram. Echocardiogram and electrocardiogram were performed 24 h after saline or LPS injections, or 26 h following CLP. FIG. 12a shows representative M-modes and 2-D images from echocardiogram in endotoxemia model. FIGS. 12h and 12j show quantitative analysis of the fractional shortenings from echocardiogram in endotoxemia model. FIGS. 12l and 12n show heart rate measurements on electrocardiogram in endotoxemia model. FIGS. 12i and 12k show quantitative analysis of the fractional shortenings from echocardiogram in CLP model. FIGS. 12m and 12o show heart rate measurements on electrocardiogram in CLP model. ANOVA was used for a statistic analysis. *; p<0.05, **; p<0.01, ***; p<0.0001.

FIGS. 13a-13f are images showing the effect of LPS administration on protein levels of E-selectin, P-selectin, ICAM-1, VCAM-1, Cox-2, and PAI-1 in control mice and mice infected with an adenoviral vector containing a gene coding for sFlt-1 in heart by immunoassay. The sFlt-ad infected mice treated with LPS show similar protein levels and expression of these proteins as compared to mice not treated with LPS.

FIGS. 13g-13l are graphs showing the effect of LPS administration on expression of E-selectin, P-selectin, ICAM-1, VCAM-1, Cox-2, and PAI-1 in control (ctrl-ad infected) mice and mice infected with an adenoviral vector containing a gene coding for sFlt-1 (sFlt-ad infected) in heart by Taqman PCR. The sFlt-ad infected mice treated with LPS show similar expression levels of these genes as compared to mice not treated with LPS.

FIGS. 14a-14f are images showing the effect of LPS administration on protein levels of E-selectin, P-selectin, ICAM-1, VCAM-1, Cox-2, and PAI-1 in control mice and mice infected with an adenoviral vector containing a gene coding for sFlt-1 in brain by immunossay. The sFlt-ad infected mice treated with LPS show similar protein levels and expression of these proteins as compared to mice not treated with LPS.

FIGS. 14g-14l are graphs showing the effect of LPS administration on expression of E-selectin, P-selectin, ICAM-1, VCAM-1, Cox-2, and PAI-1 in control (ctrl-ad infected) mice and mice infected with an adenoviral vector containing a gene coding for sFlt-1 (sFlt-ad infected) in brain by Taqman PCR. The sFlt-ad infected mice treated with LPS show similar expression levels of these genes as compared to mice not treated with LPS.

FIGS. 15a-15f are images showing the effect of LPS administration on protein levels of E-selectin, P-selectin, ICAM-1, VCAM-1, and Cox-2 in control mice and mice infected with an adenoviral vector containing a gene coding for sFlt-1 in lung by immunoassay. The sFlt-ad infected mice treated with LPS show similar protein levels and expression of these proteins as compared to mice not treated with LPS.

FIGS. 15g-15l are graphs showing the effect of LPS administration on expression of E-selectin, P-selectin, ICAM-1, VCAM-1, Cox-2, and PAI-1 in control (ctrl-ad infected) mice and mice infected with an adenoviral vector containing a gene coding for sFlt-1 (sFlt-ad infected) in lung by Taqman PCR. The sFlt-ad infected mice treated with LPS show similar expression levels of these genes as compared to mice not treated with LPS.

FIGS. 16a-16f are graphs showing sFlt-1 reduces expression E-selectin, ICAM-1, and VCAM-1 in human umbilical vein endothelial cells (HUVEC) treated with LPS in 2% mouse serum as compared to cells not treated with sFlt-1.

FIGS. 16g-16j are graphs and a table showing the effect of VEGF and PlGF on cytokine responsiveness of primary human endothelial cells. These results are of quantitative TaqMan analyses (mRNA copy number per 10⁶ copies 18S) of VCAM-1 (FIGS. 16g and 16j), Cox-2 (FIGS. 16h and 16j), and tissue factor (FIGS. 16i and 16j). FIG. 16j additionally shows E-selectin, P-selectin, ICMA-1, and PAI-1 in serum-starved HUVEC treated for 4 h in the absence (CTRL, control) or presence of TNF-α, VEGF, PlGF alone or in combination.

FIGS. 17a-17f are graphs showing that LPS induces upregulation of PlGF in heart, lung, kidney, and brain as compared to animals not treated with LPS. Treatment with sFlt-1 prevents the observed increases of PlGF upon LPS treatment.

FIGS. 18a and 18b are graphs showing that TNF and IL-1 receptor knockout mice show smaller increases in PlGF and VEGF 24 hours following treatment with 18 mg/kg LPS as compared to the increases observed in wild-type mice treated with LPS.

FIG. 19 is a set of graphs showing VEGF and PlGF protein levels in a mouse model of endotoxemia. Results of ELISA for VEGF (top) and PlGF (bottom) in tissues from mice injected with or without LPS at the time points indicated are shown. *; p<0.05, **; p<0.01, ***; p<0.0001.

FIGS. 20a-20i are graphs showing the effect of sFlt-1 overexpression on circulating cytokine levels and D-dimers in mouse model of endotoxemia. FIGS. 20a-20f show mice injected with adenovirus overexpressing GFP (CTRL-ad) or sFlt-1 (sFlt-ad). Three days later, the animals were administered saline (control) or LPS IP. Serum or plasma levels of TNF-α, IL-1α, IL-1β, IL-6, sFlt-1 and D-dimer were measured at 6, 12 and/or 24 h. n.s., not significant. *; p<0.05, **; p<0.01, ***; p<0.0001. FIGS. 20g-20i show circulating levels of VEGF and IL-6 levels in triple mutant mice (IL-1−/−, TNFR1−/−, TNFR2−/−) with LPS-induced endotoxemia. Plasma levels of VEGF, PlGF, and IL-6 in LPS-injected wild type (WT) or triple mutant (TM) mice 24 h following IP injection of saline (control) or LPS. *; p<0.05, **; p<0.01, ***; p<0.0001.

### DETAILED DESCRIPTION

The present invention includes methods of diagnosing and treating an inflammatory response such as sepsis, severe sepsis, or septic shock. The invention further includes methods of screening for candidate compounds useful in the treatment of inflammatory response. These methods stem from the discovery that circulating levels of VEGF, PlGF, and sFlt-1 are elevated in sepsis in a time dependent manner in animal and human models of sepsis, the increased levels of VEGF and PlGF are linked to the pathology of sepsis, and treatment with sFlt-1 decreases the severity of or prevents sepsis, severe sepsis, and septic shock.

Specifically, Adenovirus (Ad)-mediated overexpression of sFlt-1 in a mouse model of endotoxemia attenuates the rise in VEGF and PlGF levels and blocks the effect of endotoxemia on cardiac function, vascular permeability, and mortality. Similarly, in a cecal ligation puncture (CLP) model, Ad-sFlt-1 protects against cardiac dysfunction and mortality. When administered in a therapeutic regimen beginning one hour after onset of endotoxemia or CLP, sFLT peptide administration results in marked improvement in cardiac physiology and survival. Systemic administration of antibodies against Flk-1, but not Flt-1, protects against sepsis mortality. Ad-mediated overexpression of VEGF, but not PlGF exacerbates the lipopolysaccharide-mediated toxic effects. Together, these data support a pathophysiological role for VEGF in mediating the sepsis phenotype.

In addition to its role in promoting endothelial permeability and proliferation, VEGF may contribute to inflammation and coagulation. For example, under in vitro conditions, VEGF induces the expression of cell adhesion molecules (E-selectin, intercellular adhesion molecule (ICAM)-1, and vascular cell adhesion molecule (VCAM)-1 in endothelial cells and promotes adhesion of leukocytes (Kim et al., J Biol Chem 276:7614-7620, 2001; Reinders et al., J Clin Invest 112:1655-1665, 2003). Moreover, VEGF signaling upregulates tissue factor mRNA, protein and procoagulant activity (Lucerna et al., J Biol Chem 278:11433-11440, 2003). These proinflammatory/procoagulant effects of VEGF are mediated, at least in part, by activation of NF-κB, Egr-1, and NF-AT transcription factors. VEGF has been implicated as a pathophysiological mediator in several human disease states, including rheumatoid arthritis, cancer, and inflammatory bowel disease (Kuenen et al., Arterioscler Thromb Vasc Biol 22:1500-1505, 2002; Harada et al., Scand J Rheumatol 27:377-380, 1998; Taha et al., Dig Dis Sci 49:109-115, 2004). Two independent studies report an association between human severe sepsis/septic shock and elevated circulating levels of VEGF (van der Flier et al., Shock 23:35-38, 2005; Pickkers et al., Shock 24:508-512, 2005). The current work was the first to identify VEGF as playing a pathogenic role in mediating the sepsis phenotype, thus representing a novel therapeutic target.

Using a number of different animal and human models, we have discovered that sepsis is associated with increased circulating levels of VEGF and PlGF. Levels were highest in endotoxemia models, intermediate in CLP, and lowest in pneumonia. Importantly, the findings were confirmed in human models of endotoxemia and severe sepsis. In all cases, induction of VEGF and PlGF levels occurred at a later time point, compared with TNF-α, IL-1 and IL-6. sFlt-1 blocked free VEGF and PlGF, but had no effect on the early increase of TNF-α, IL-1β and IL-6. Moreover, triple mutant mice that lack the ability to respond to IL-1 and TNF-α did not demonstrate LPS-mediated increases in VEGF and PlGF levels. Together, these findings indicate that VEGF and PlGF are late markers of sepsis, and lie downstream of early response cytokines.

The administration of sFlt-1 attenuated LPS- and CLP-mediated morbidity and mortality. Although sFlt-1 binds both VEGF and PlGF, several findings point to the importance of VEGF in mediating sepsis pathophysiology. First, PlGF concentrations were at least 10-fold lower than circulating VEGF concentrations in human sepsis. Second, PlGF binds to Flt-1 at a lower affinity than VEGF. Third, VEGF but not PlGF, sensitized endothelial cells to the effects of low TNF-α concentrations. Fourth, overexpression of VEGF, but not PlGF, resulted in marked increase in LPS sensitivity (100% mortality). Finally, and most importantly, antibodies against Flk-1, but not Flt-1, attenuated sepsis mortality.

All organs examined displayed increased levels of PlGF protein, while the heart, liver and kidney were the major sources of VEGF induction. The finding that VEGF levels were similar in serum and plasma (both in control and LPS-treated mice) argues against a significant contribution of platelets to the circulating pool of VEGF. Although hypoxia is known to induce the expression of both growth factors, hypoxemia is not a universal finding in mouse and human models of sepsis. Studies have shown that inflammatory mediators, including IL-1, IL-6, and Cox, may increase mRNA expression of VEGF in various cell types (Stocks et al., FEBS Lett 579:2551-2556, 2005; Jung et al., Angiogenesis 4:155-162, 2001; Loeffler et al., Int J Cancer 115:202-213, 2005). Under in vitro conditions, VEGF and glucose have been shown to induce PlGF mRNA and protein levels in endothelial cells (Zhao et al., Microvasc Res 68:239-246, 2004). Thus, the cytokine storm associated with sepsis may contribute to the increase in VEGF and PlGF levels.

The observation that VEGF sensitizes endothelial cells to the effects of low TNF-α concentrations suggests that the high VEGF levels in sepsis may accentuate the activation phenotype. In addition, VEGF induction of endothelial permeability may contribute to the morbidity and mortality in sepsis.

Our finding that endotoxemia is associated with increased circulating levels of sFlt-1 is novel. Previous studies have demonstrated that circulating levels of sFlt-1 are increased in the third trimester of pregnancy, and are abnormally elevated in patients with preeclampsia (Maynard et al., J Clin Invest 111:649-658, 2003). These latter changes are associated with a reduction in circulating free VEGF and PlGF. Interestingly, TNF-α has been shown to induce release of sFlt-1 from normal placental villous explants (Ahmad et al., Circ Res 95:884-891, 2004). Together, sepsis-associated changes in circulating sFlt-1 may represent an endogenous compensatory antiinflammatory mechanism.

In summary, we have identified an association between sepsis and increased circulating levels of VEGF, PlGF, and sFlt-1. More importantly, the results suggest a pathophysiological role for VEGF in mediating the sepsis phenotype, and indicate that VEGF, PlGF, and sFl-t can be useful in both diagnostic/prognostic assays, as well as therapeutically in treating inflammation disorders such as sepsis.

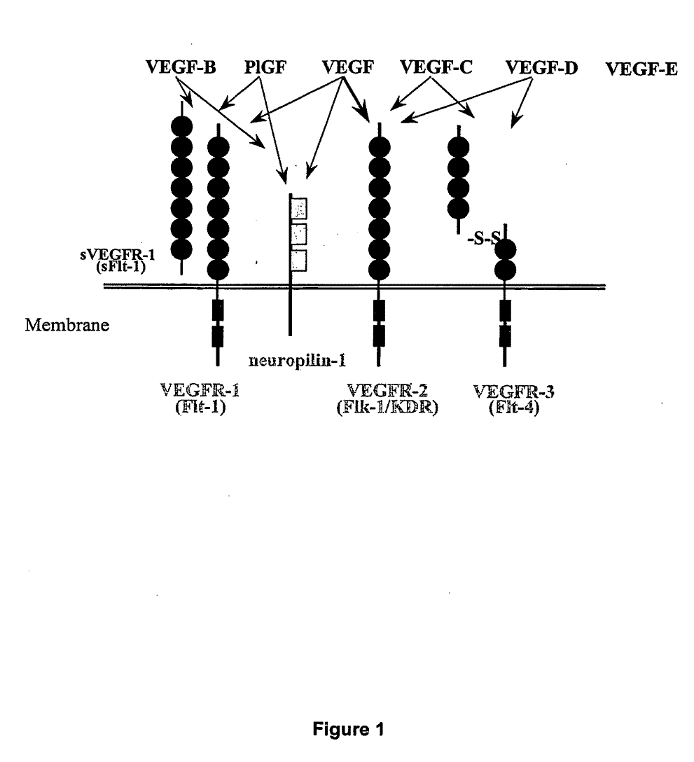

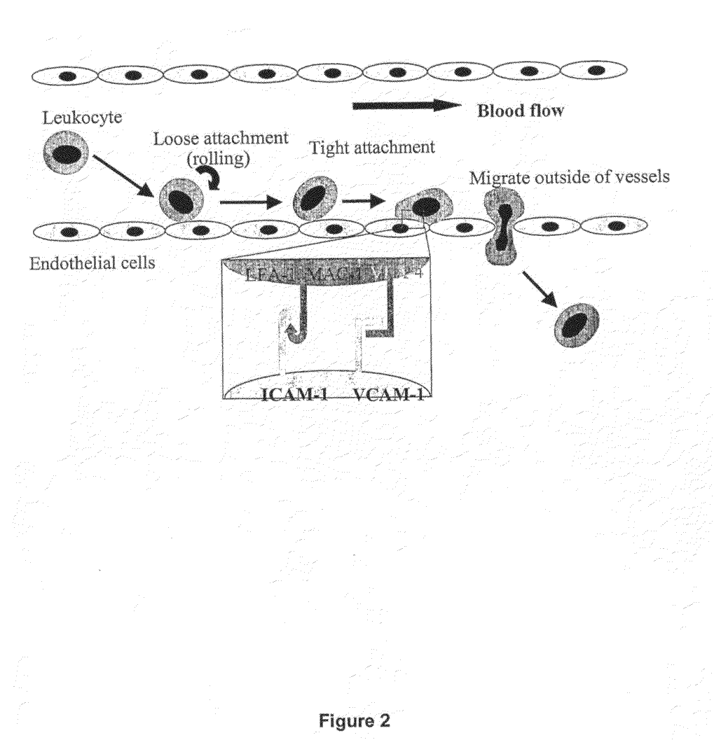

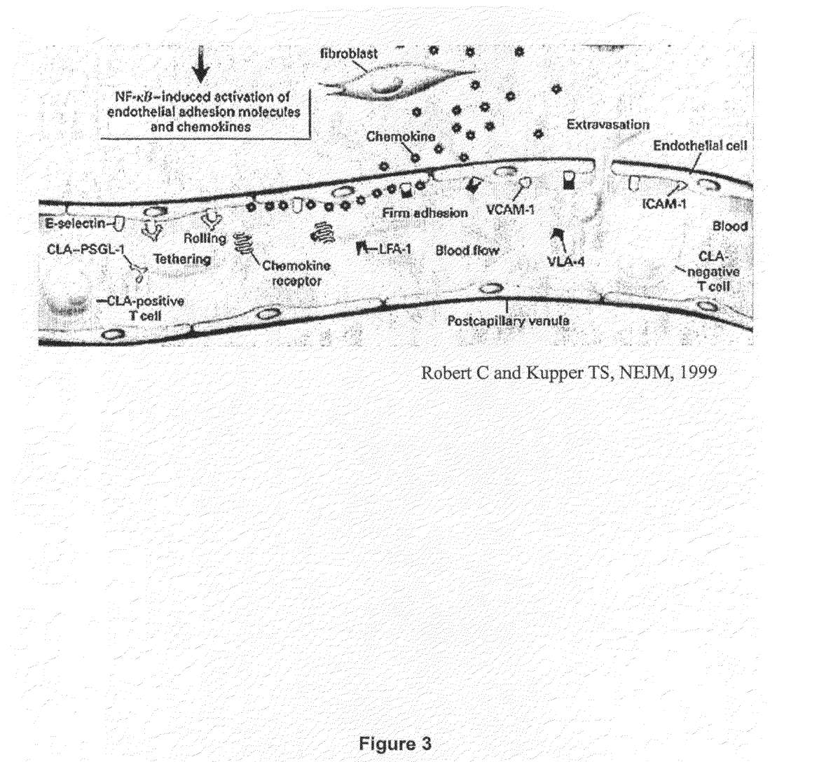

VEGF, its variants (e.g., VEGF-A, VEGF-B, VEGF-C, VEGF-D, and VEGF-E), and PlGF interact with membrane receptors VEGFR-1 (Flt-1), VEGFR-2 (Flk-1/KDR), VEGFR-3 (Flt-4), and neuropilin-1 as well as soluble forms of these receptors (e.g., sFlt-1; see FIG. 1) are involved in inflammatory response. The NF-κ induced response is shown in FIG. 2, and in greater detail in FIG. 3.

The following examples are meant to illustrate the invention and should not be construed as limiting.

### Example 1

### sFlt-1, VEGF, and PlGF and Inflammatory Response

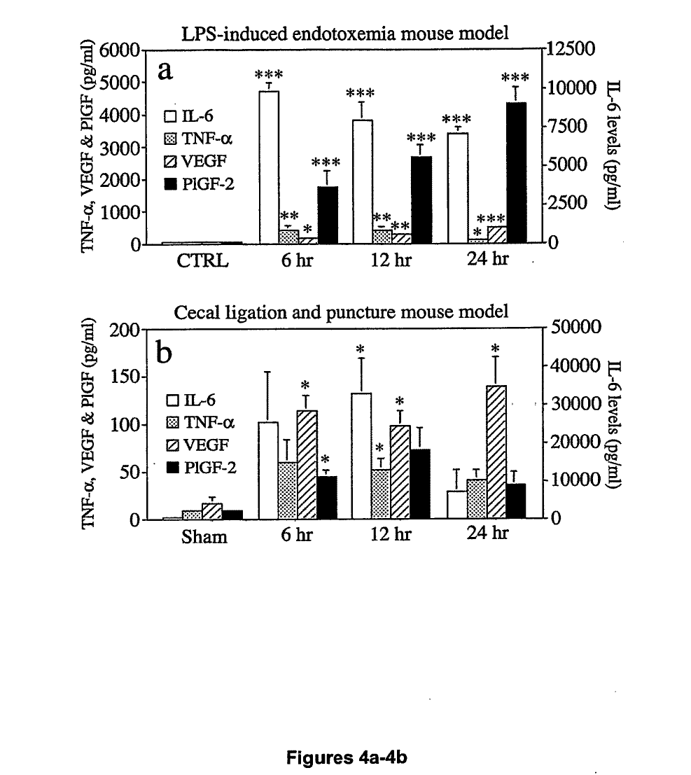

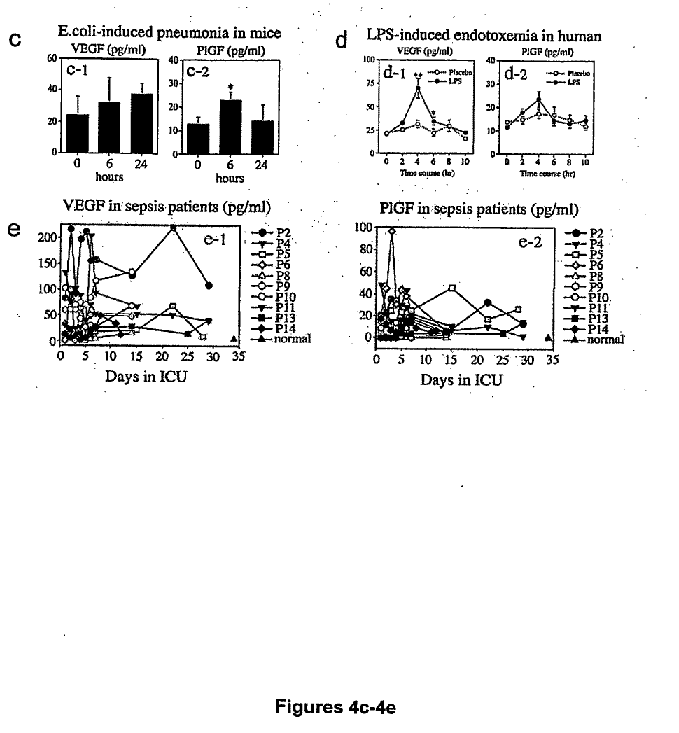

A pronounced upregulation of plasma VEGF and PlGF levels in both the lipopolysaccharide (LPS)-induced endotoxemia mouse model and the cecal ligation puncture (CLP) mouse model is observed (FIGS. 4a-4b). Intraperitoneal administration of lipopolysaccharide (LPS) in mice resulted in a time-dependent increase in plasma VEGF and PlGF concentrations, with peak levels (477 pg/ml and 4311 pg/ml, respectively) occurring at 24 h (FIG. 4a). By contrast, circulating levels of IL-6 and TNF-α were maximal at the earliest time point measured (6 h). In a CLP model of sepsis, peak levels of VEGF (137.26 pg/ml) and PlGF (71.25 pg/ml) occurred at 24 h and 12 h, respectively (FIG. 4b). In a mouse model of Escherichia coli pneumonia, plasma VEGF levels were not significantly altered, whereas PlGF levels were increased (23.01 pg/ml) at 6 h (FIG. 4c).

Human Data

In human subjects, the systemic administration of LPS resulted in elevated circulating levels of VEGF and PlGF (FIG. 4d), with peak levels (70 pg/ml and 23.5 pg/ml, respectively) occurring at 4 h, in contrast to TNF-α, and IL-6, which peaked at 1.5 and 2.5 h, respectively (data not shown). Plasma levels of VEGF and PlGF were measured in 10 patients with severe sepsis and 10 healthy volunteers. At study entry, VEGF levels in the patients (mean and SD=46.49±46.17 pg/ml) were significantly higher than in the healthy volunteers (mean and SD=3.83±3.16 pg/ml) (P=0.009). Similarly, PlGF levels in the patients at study entry (mean and SD=13.52±14.55 pg/ml) were significantly higher compared with healthy volunteers (mean and SD=0.18±0.58 pg/ml) (P=0.009). In most cases (8/10), the VEGF and PlGF levels remained elevated during their ICU stay (in some cases, up to 29 days). The maximum VEGF and PlGF levels were 367 pg/ml and 96 pg/ml, respectively (FIG. 4e).

VEGF Levels Correlates with Severity

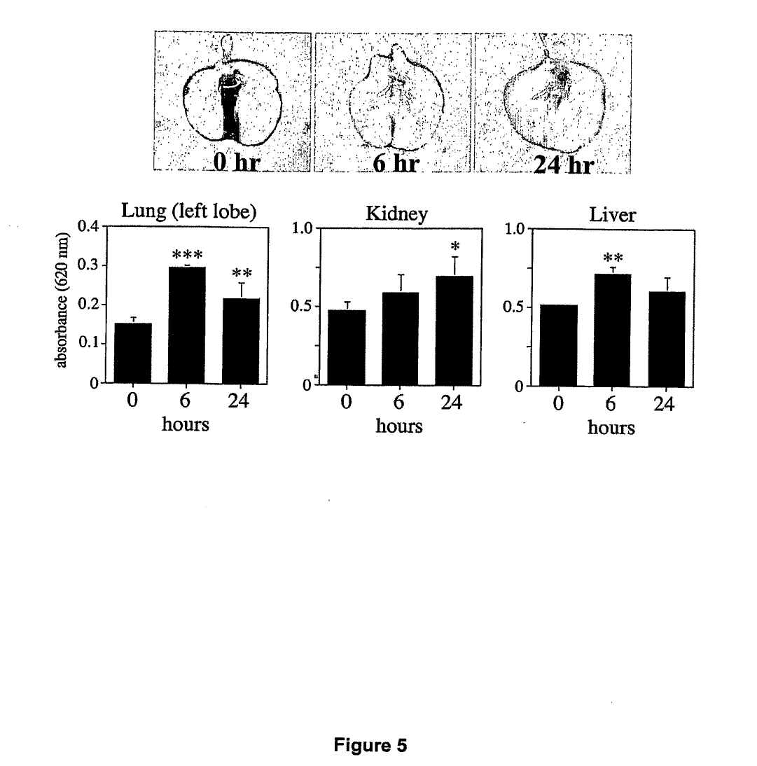

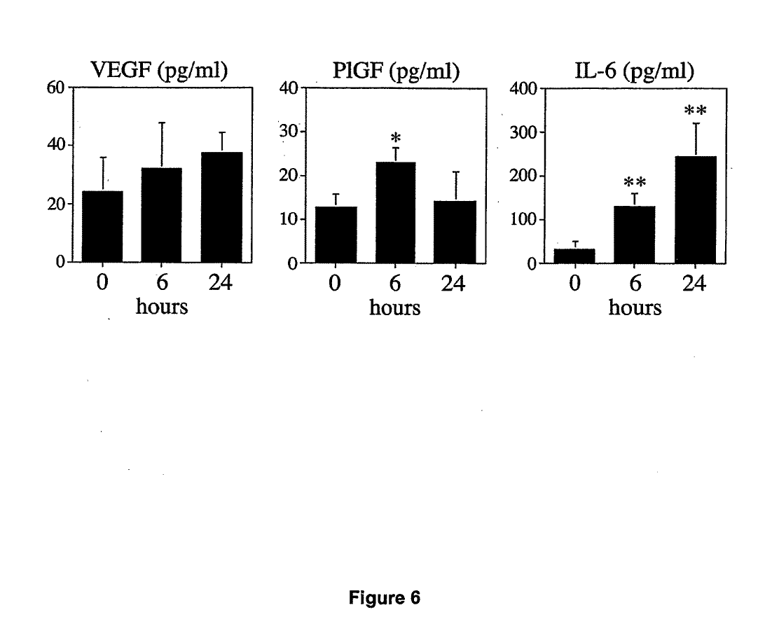

VEGF levels are significantly correlated with the severity of endotoxemia. The mile assay shows a significant vascular leakage in lung, liver and kidney tissues following lung infection, as compared with uninfected mice (FIG. 5). PlGF and IL-6 are both increased by lung infection, as compared to uninfected mice (FIG. 6), indicating a link between PlGF and inflammatory response. Physiological measurements in an endotoxemia model revealed reduced cardiac output, blood pressure, and body temperature as compared with age-matched control mice. These results indicate that detection of VEGF and PlGF in a subject is diagnostic of sepsis and that the severity of sepsis is correlated to the amount of VEGF or PlGF detected in the subject.

sFLt-Treated Mice

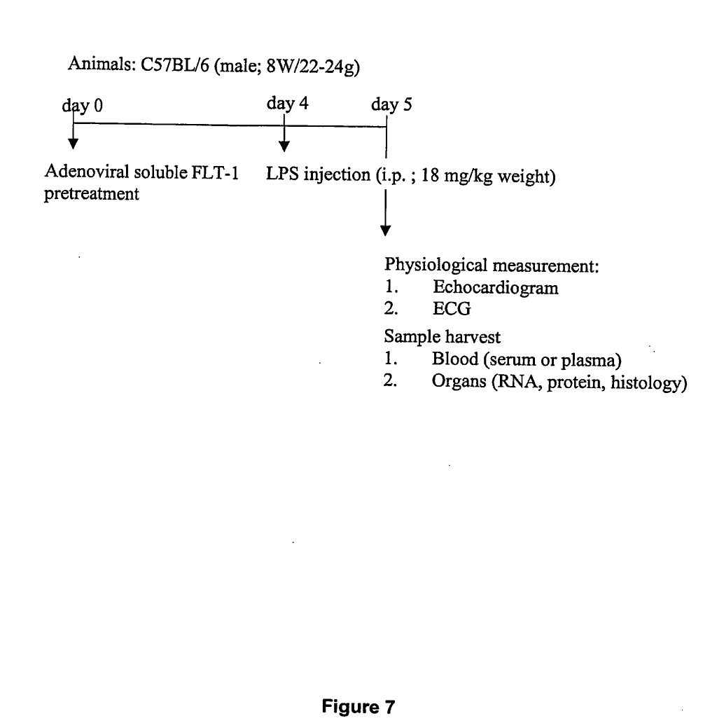

Systemic blockade of circulating VEGF by soluble Flt-1 (sFlt-1) protein prevents LPS-induced endotoxemia symptoms was demonstrated using the following procedure (FIG. 7). sFlt-1 is a natural splice variant of the cell surface receptor Flt-1 that binds to free VEGF-A, VEGF-B and PlGF, thus blocking their interaction with cell surface receptors (Kendall et al., Biochem Biophys Res Commun 226:324-328, 1996). Previous studies have shown that Ad-sFlt-1 transduces liver hepatocytes in vivo and results in high circulating levels of sFlt-1 for up to 2 weeks following injection (Kuo et al., Proc Natl Acad Sci USA 98:4605-4610, 2001; Maynard et al., J Clin Invest 111:649-658, 2003).

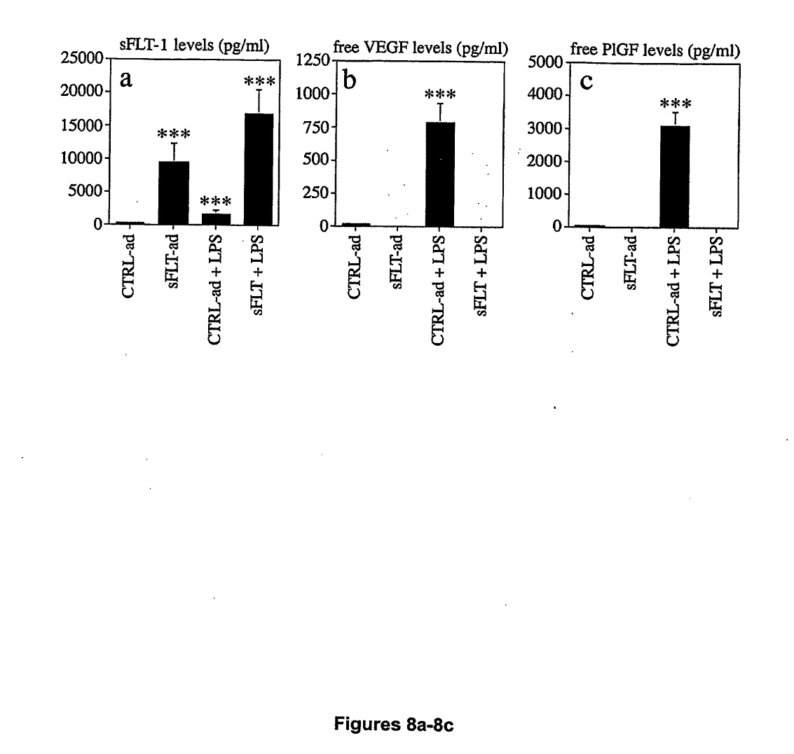

Mice were infected with an adenoviral vector containing a gene coding for sFlt-1 (sFlt-ad) or a control gene (ctrl-ad). Four days later, the mice were administered an intraperitoneal (i.p.) injection of 18 mg/kg LPS. One day later (five days following infection with the adenoviral vector; see FIG. 7), the mice were evaluated using echocardiogram and ECG, and samples from the mice, including blood (e.g., serum and plasma) and organs (e.g., brain, heart, liver, lung, spleen, kidney) were harvested and analyzed. As shown in FIGS. 8a-8f, Ad-sFlt-1-treated mice displayed elevated levels of sFlt-1 in the serum, as compared to Ad-GFP-treated animals. Importantly, Ad-sFlt-1 significantly inhibited LPS-mediated induction of free VEGF and PlGF, whereas control Ad had no such effect. In control animals, LPS increased circulating levels of endogenous sFlt-1 (from 159.62 pg/ml to 1585.40 pg/ml), and further increased total sFLT-1 in Ad-sFlt-1-treated animals. In summary, infection with sFlt-ad resulted nearly complete blockage of free VEGF and PlGF following LPS administration as compared to ctrl-ad infected mice.

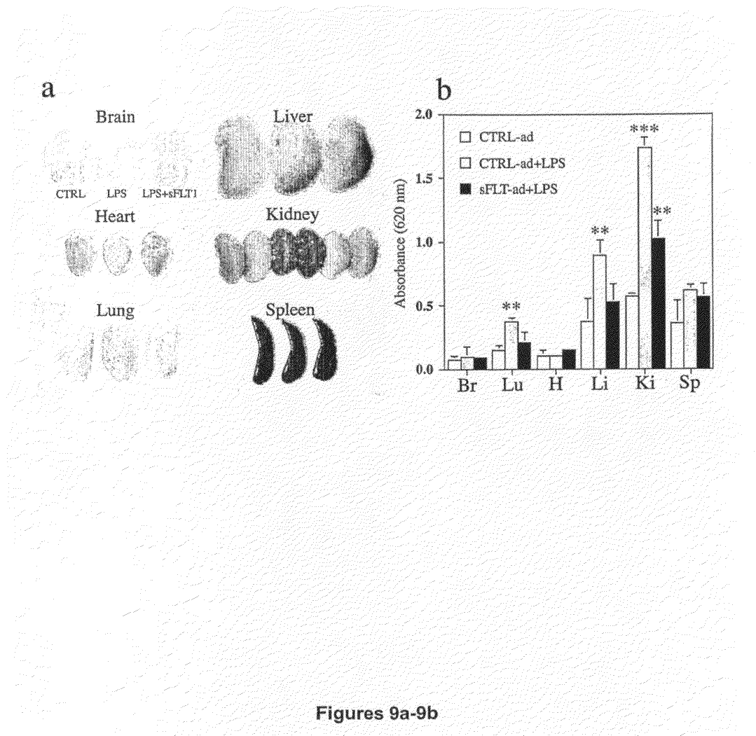

sFlt-ad infected mice were also protected against vascular leakage induced by LPS administration in lung, liver, and kidney as compared to ctrl-ad infected mice (FIGS. 9a and 9b). LPS administration resulted in organ-specific loss of barrier function, with increased extravasation of Evans blue dye in the lungs, liver and kidney, but not in brain, heart or spleen (FIGS. 9a and 9b). Over-expression of sFlt-1 completely blocked LPS-induced vascular leak in the liver and lung, but only partially inhibited the effect of LPS on kidney permeability.

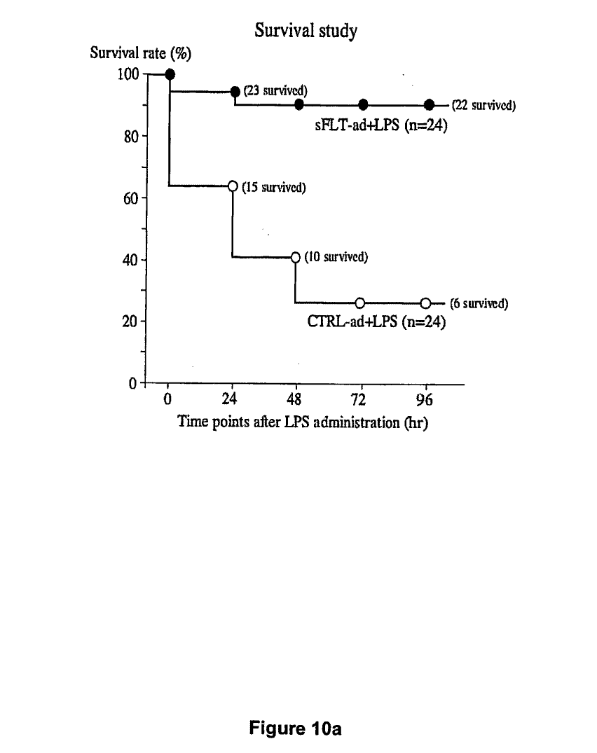

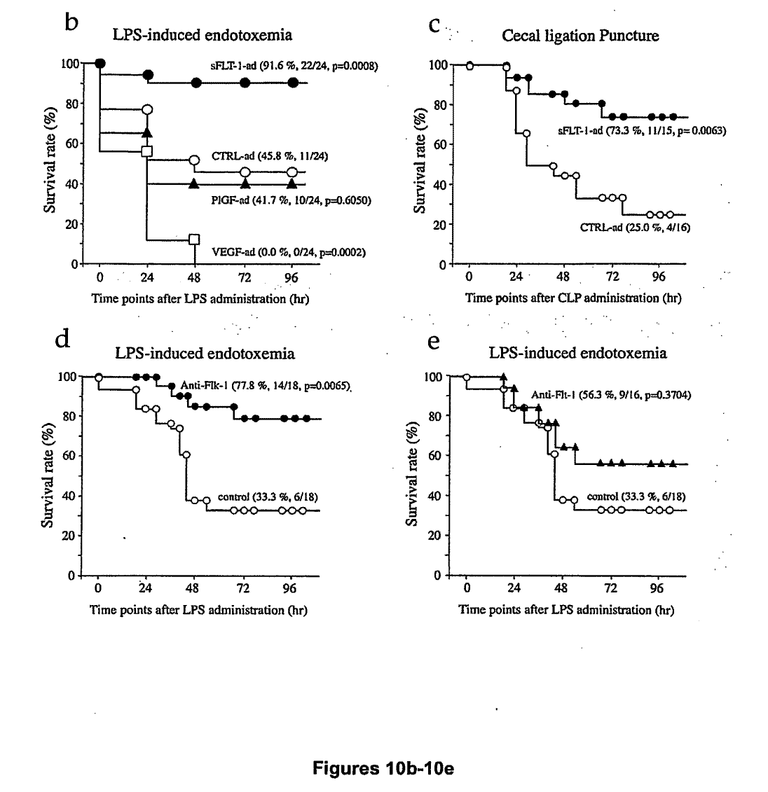

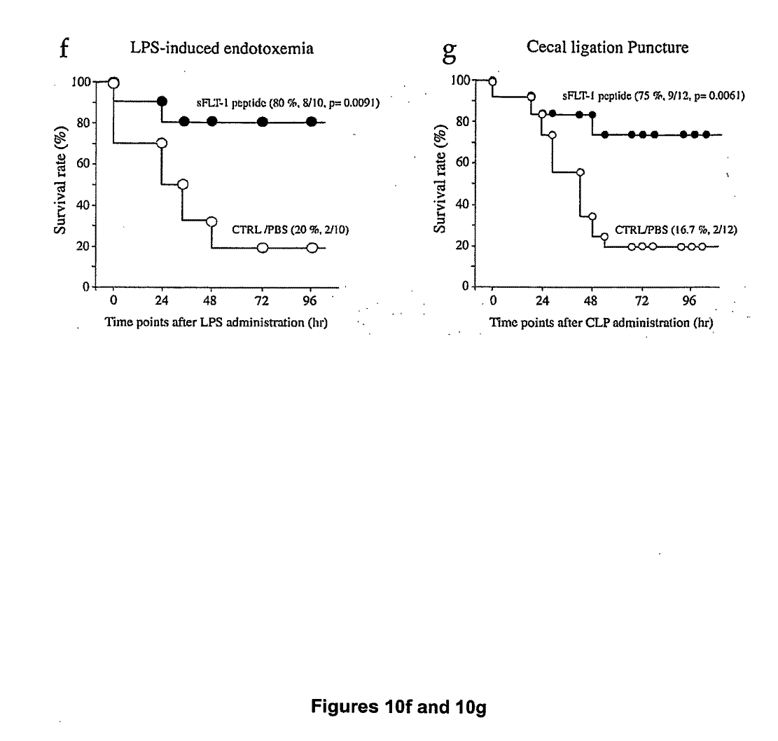

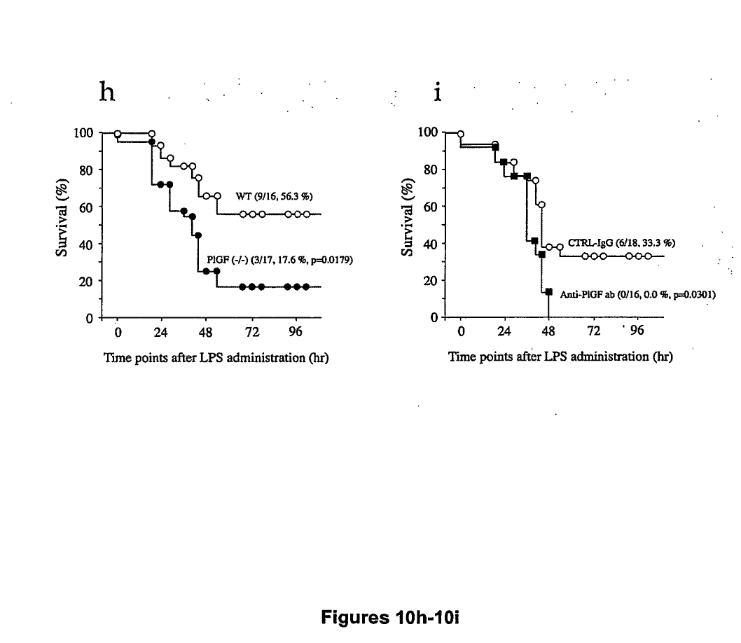

The sFlt-ad infected mice showed substantially reduced mortality upon LPS treatment as compared to ctrl-ad infected mice (FIG. 10a). Twenty-two of 24 of the sFlt-ad mice survived 96 hours following LPS treatment whereas only six of the 24 ctrl-ad infected mice survived. The sFlt-ad infected mice also showed substantial decreases in D-dimer (FIG. 11a), which measures the tendency to form blood clots, and IL-6 (FIG. 11b), serum levels of which are increased in inflammatory response, as compared to ctrl-ad infected mice.

Further, survival studies were carried out in LPS-treated (20 mg/kg) mice overexpressing GFP (control), VEGF, PlGF, or sFlt (FIG. 10b). In the control group, 13/24 (54.2%) mice died from endotoxemia. As noted above, sFlt-1 resulted in significant reduction in mortality (2 of 24 animals died). Overexpression of VEGF (mean plasma levels 5.08 ng/ml) resulted in a marked increase in LPS sensitivity (100% mortality), whereas over-expression of PlGF (mean plasma levels 28.23 ng/ml) had no such effect. sFlt-1 also improved survival in the CLP model of sepsis (FIG. 10c). A total of 4 of 15 (27%) mice overexpressing sFlt-1 died from CLP-induced sepsis, compared with 12 of 16 (75%) in the GFP-expressing control group (p=0.0063) (FIG. 10c). To confirm a primary role for VEGF, animals were pretreated with neutralizing antibodies against Flt-1 or Flk-1. In these experiments, anti-Flk-1 antibodies, but not anti-Flt-1 antibodies, reduced mortality in the mouse endotoxemia model (FIGS. 10d and 10e, respectively). Together, these findings suggest that VEGF-A is a critical determinant of the sepsis phenotype.

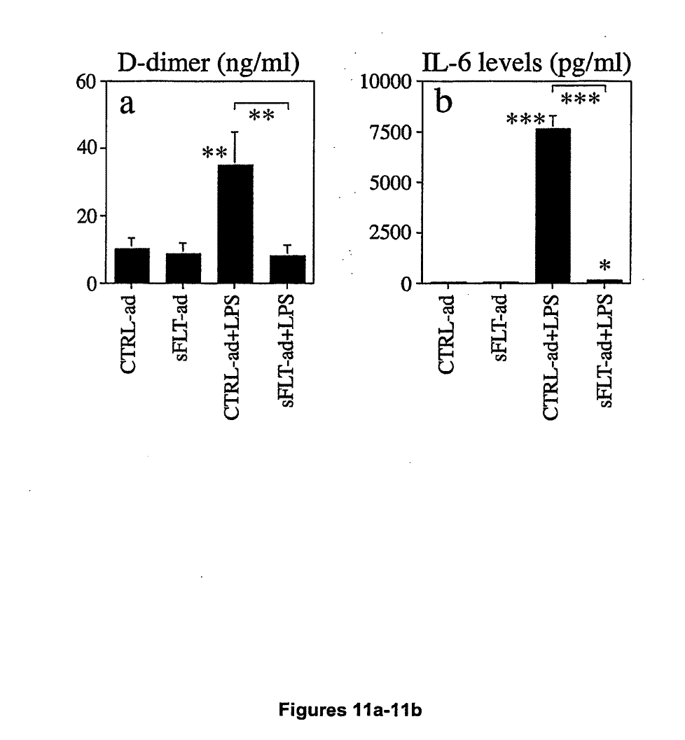

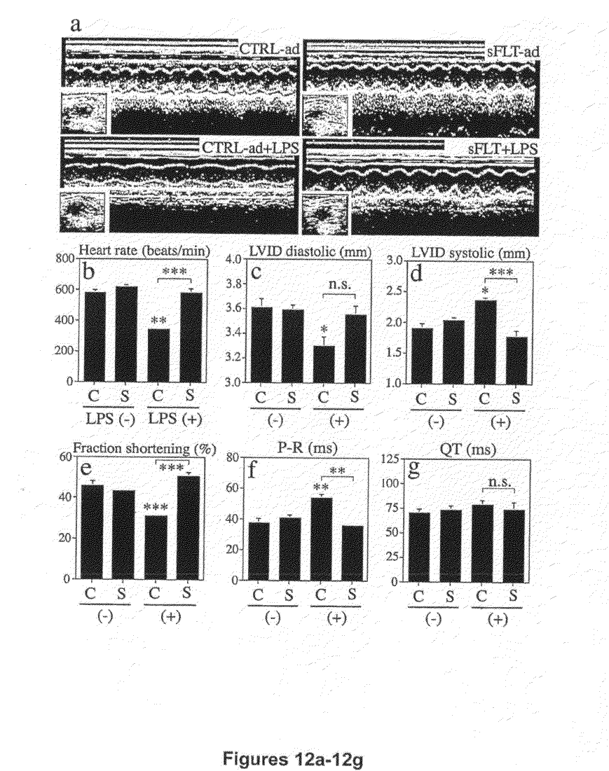

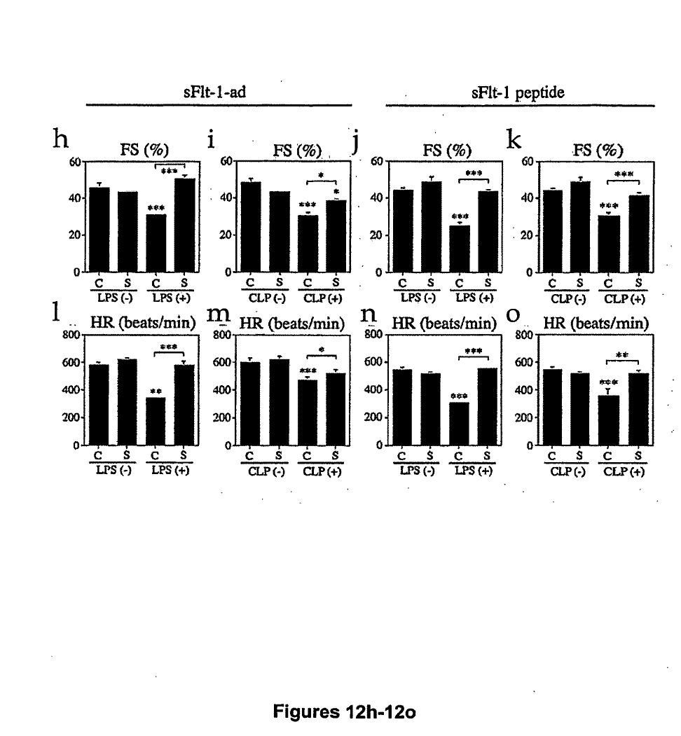

To determine whether VEGF inhibition following onset of sepsis results in improved outcome, mice were injected intravenously with 1 μg of human recombinant soluble Flt-1 peptide or equal volume PBS (control) every 3 h (×4 doses) beginning 1 h following LPS administration or CLP procedure. Soluble Flt-1 peptide completely blocked the effects of LPS or CLP on fractional shortening (as shown in FIGS. 12j and 12k) and heart rate (FIGS. 12n and 12o), as described below. In survival studies, recombinant sFlt-1 improved survival in both endotoxemia and CLP models of sepsis. Mortality in LPS was reduced from 80% to 20% (p=0.0091) (FIG. 10f), while CLP-mediated mortality was decreased from 75% to 16.7% (p=0.0061) (FIG. 10g). These results indicate that blocking VEGF function (e.g., using sFLt-1) may be useful for treatment of inflammatory disorders such as sepsis, severe sepsis, or septic shock.

Systemic blockade of VEGF (via infection of sFlt-ad) prevents LPS-induced morbidity in mice, as measured by cardiac output, blood pressure, and body temperature (see FIGS. 12a-12g). Systemic administration of LPS or CLP in mice resulted in marked depression of cardiac function (FIGS. 12a-12o), as evidenced by reduced fractional shortening (FIGS. 12h-k) and heart rate (FIGS. 12l-o) on echocardiography, and was associated with PR interval prolongation on electrocardiogram (FIG. 12f). These effects were completely blocked by Ad-mediated over-expression of sFlt-1 (FIGS. 12h and 12l), but not GFP (control) or PBS (control) injection. Similar results were demonstrated in the CLP model (FIGS. 12i and 12m).

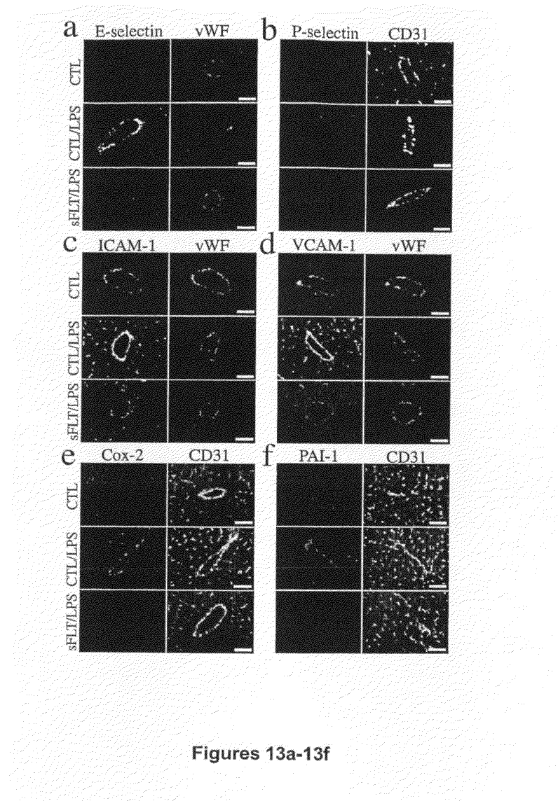

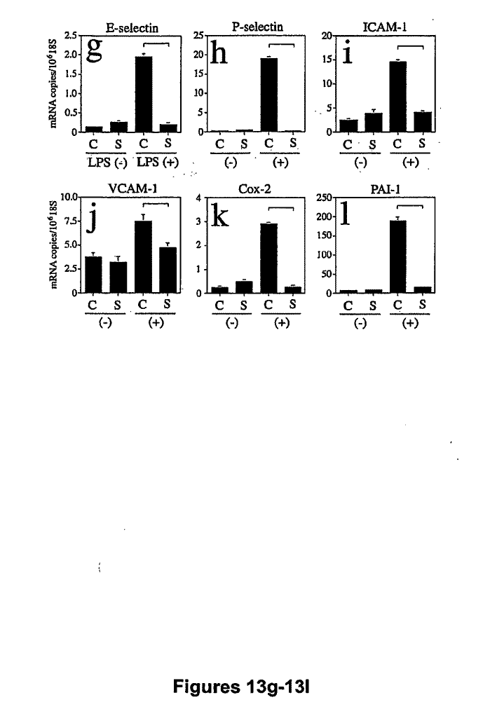

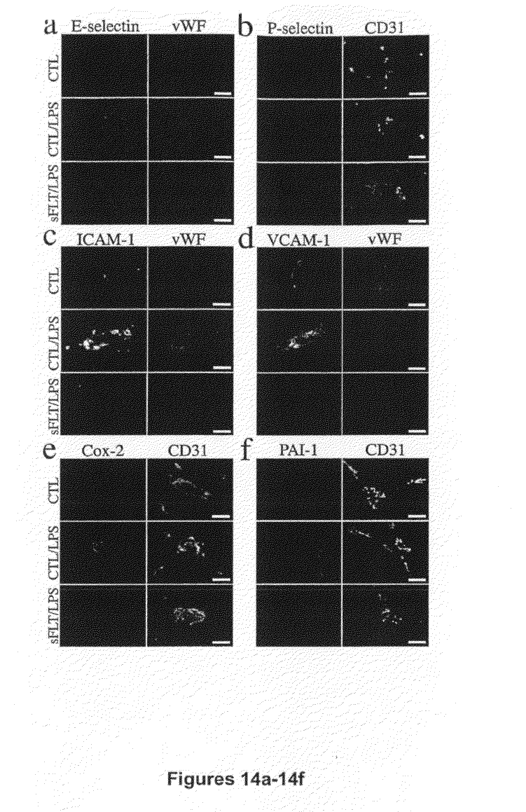

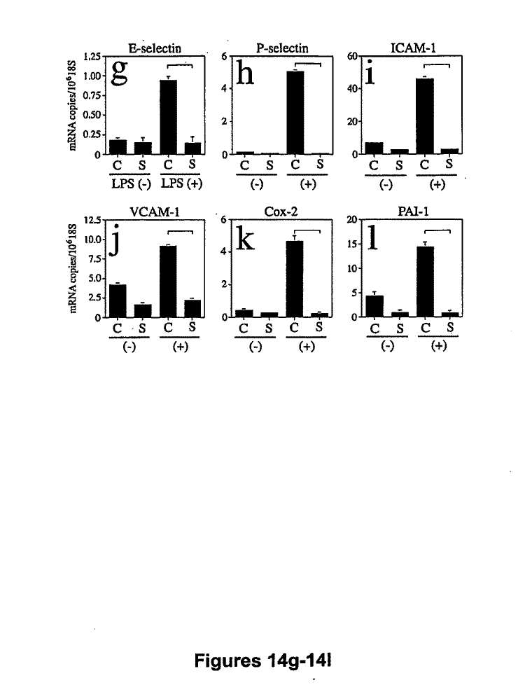

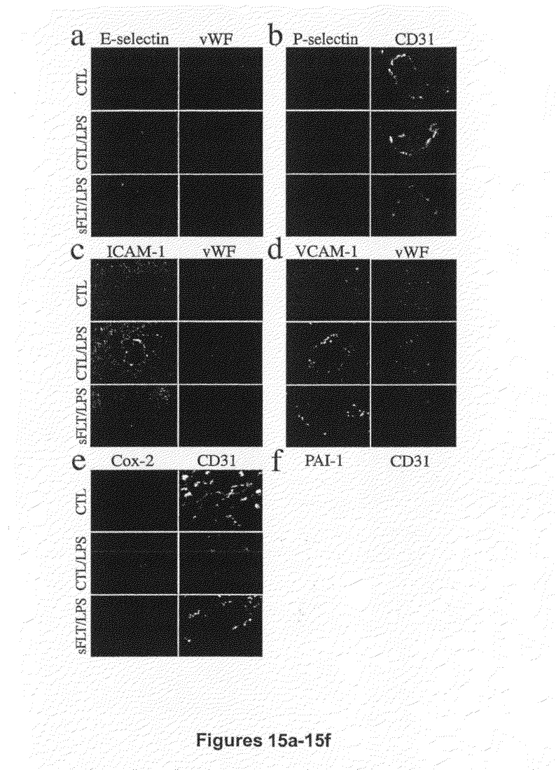

Endotoxemia in mice was associated with increased mRNA expression of inflammatory and procoagulant molecules (FIGS. 13g-13l, 14g-14l, and 15g-15l). As a negative control, VE-cadherin mRNA levels did not increase in any tissue examined (data not shown). In immunofluorescent studies, most of these inflammatory mediators were localized primarily in the endothelium. Infection of sFlt-ad in mice also decreases the expression (as measured by Taqman PCR) and protein levels (as measured by immunofluorescence) of markers of inflammation (e.g., E-selectin, P-selectin, ICAM-1, VCAM-1, Cox-2, and PAI-I) in mice treated with LPS to levels similar to those observed in control (both uninfected and ctrl-ad infected) mice not treated with LPS. These reductions are observed systemically, including heart (FIGS. 13a-13l), brain (FIGS. 14a-14l), and lung (FIGS. 15a-15l). In the heart, E-selectin, P-selectin, ICAM-1, VCAM-1, Cox-2 were induced in small venules. ICAM-1 and VCAM-1 were also increased in capillary endothelium. PAI-1 was induced in capillaries alone. In the brain, all of the above mediators were induced in endothelium of venules, but not capillaries. In the lung, E-selectin, P-selectin, ICAM-1, VCAM-1, and Cox-2 were increased in venular endothelium. ICAM-1 and PAI-1 were increased in lung parenchyma. These immunohistochemical analyses demonstrated that the endothelial expression of these activation markers during endotoxemia was abrogated by the administration of sFlt-1.

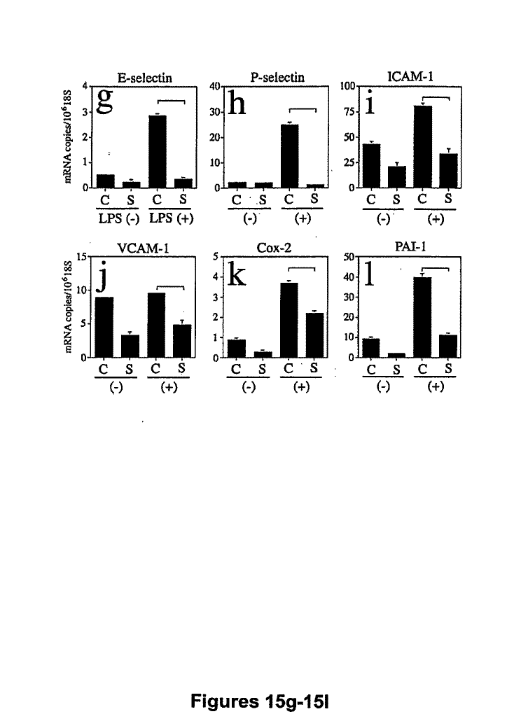

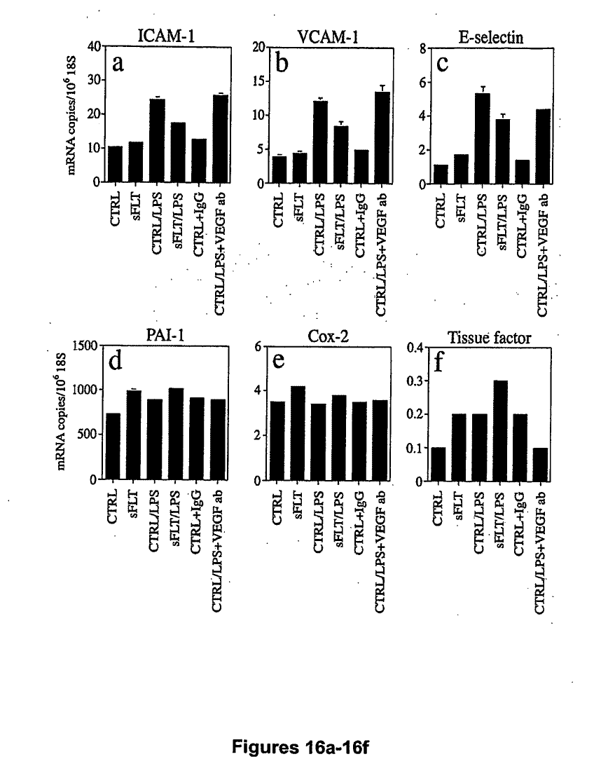

Similar results are observed in cell culture models. Human umbilical vein endothelial cells (HUVEC) treated with LPS show increases in E-selectin, ICAM-1 and VCAM-1 as compared to control cells. These increases are reversed by transfecting the HUVEC with sFlt-1 (FIGS. 16a-16f). To gain further insights into the mechanisms by which VEGF mediates sepsis morbidity and mortality, HUVEC were incubated for 4 h with TNF-α, VEGF and PlGF, alone or in combination. VEGF, but not PlGF, induced the expression of several genes, including VCAM-1 (9.3-fold) and tissue factor (16.4-fold) (see FIGS. 16h-16j). Endothelial cells treated with 0.1 ng/ml TNF-α alone demonstrated 29.4-fold increase in VCAM-1, 2.13-fold increase in TF, and no change in Cox-2 mRNA levels. In contrast, 0.1 ng/ml TNF-α plus 100 ng/ml VEGF resulted in significantly increased expression of VCAM-1, Cox-2, and tissue factor over and above the effects of VEGF alone. VEGF may therefore sensitize endothelial cells to low concentrations of TNF-α.

PlGF is Upregulated after LPS Treatment

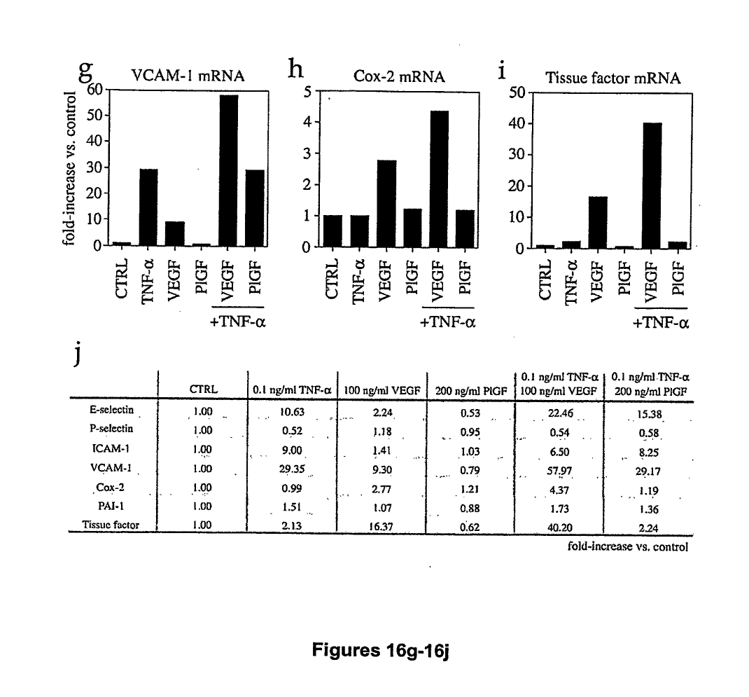

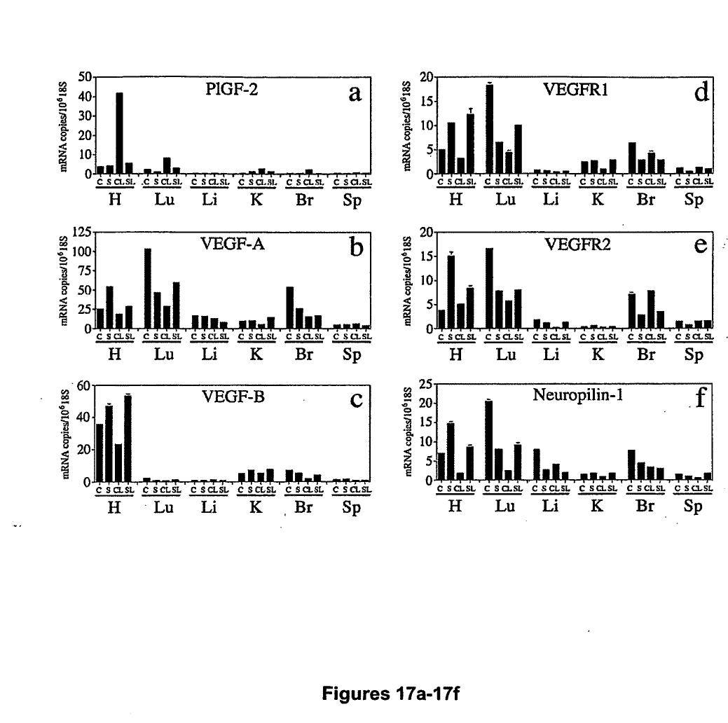

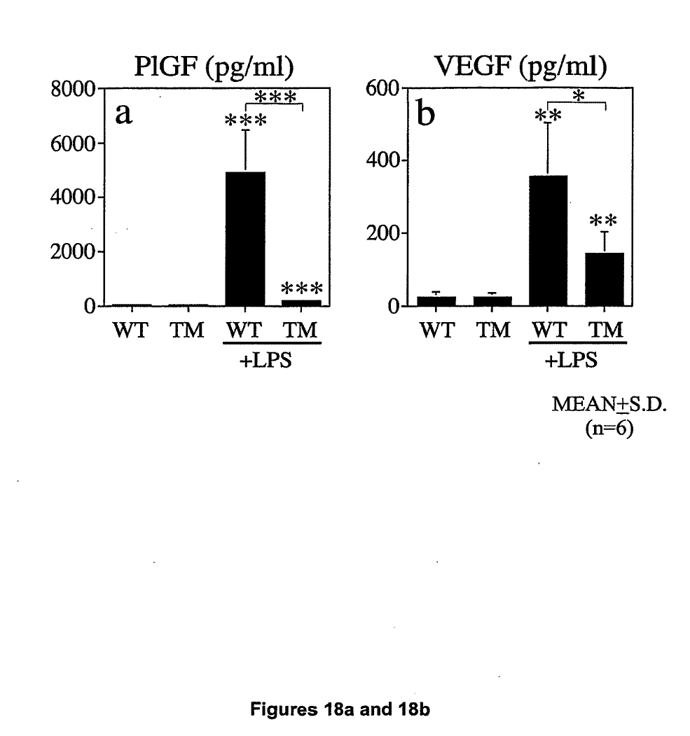

Upregulation of PlGF is also observed in heart, lung, kidney, and brain after LPS treatment, an increase normalized by sFlt-1 treatment (FIGS. 17a-17f). TNF and IL-11 receptors knockout mice treated with LPS also have levels of VEGF and PlGF similar to mice (both wild-type and TNF/IL-1 receptor knockout mice) untreated with LPS, whereas LPS treated wild-type mice exhibit increase in both VEGF and PlGF (FIGS. 18a and 18b).

Loss of PlGF Activity Results in Increased Mortality Rates LPS-Treated Mice

The effects of an anti-PlGF antibody and genetic deficiency of PlGF on sepsis mortality were also evaluated on survival. Studies were performed in 17 mg/kg LPS-treated wild-type mice and PlGF null mice, and in 20 mg/kg LPS-treated mice with control IgG or anti-PlGF antibody. In wildtype mice, 7/16 (43.7%) mice died from endotoxemia (FIG. 10h). PlGF deficiency resulted in a significant increase in mortality (14/17 animal died; p=0.0179). Similarly, anti-PlGF antibody treatment resulted in marked increase in mortality (100% mortality; p=0.0301) as compared with control IgG treated group, 12/18 (66.7%) mice died from LPS-induced endotoxemia (FIG. 10i).

Source of Increased VEGF and PlGF

To determine the source of elevated growth factor concentrations, VEGF and PlGF levels were assayed in various tissues using ELISA. Systemic administration of LPS in mice resulted in increased VEGF protein levels at 6 h in liver (3.8 fold), kidney (1.9 fold), and heart (3.7 fold), but decreased levels in brain, lung, and spleen (FIG. 19). PlGF protein was increased in all tissues examined, including brain (4.8 fold), lung (7.1 fold), heart (71.8-fold), liver (35.2 fold), kidney (28.5 fold), and spleen (28.4 fold) (FIG. 19). There were no differences in VEGF protein in the serum compared with plasma of LPS-treated or control mice, arguing against a significant contribution of platelets to the plasma VEGF levels in endotoxemia (data not shown). Based on these findings, sepsis is clearly associated with increased expression and circulating levels of VEGF and PlGF.

Effects of Cytokine Induction by sFlt-1

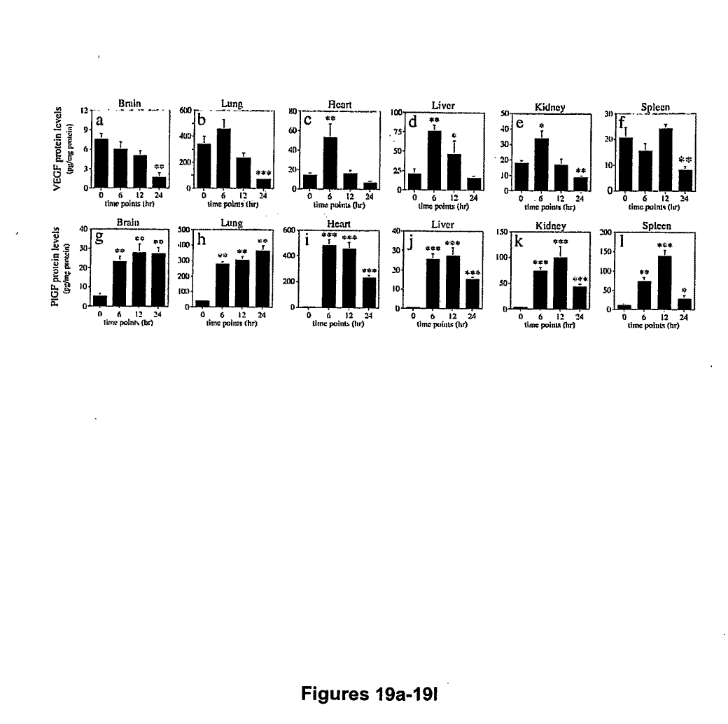

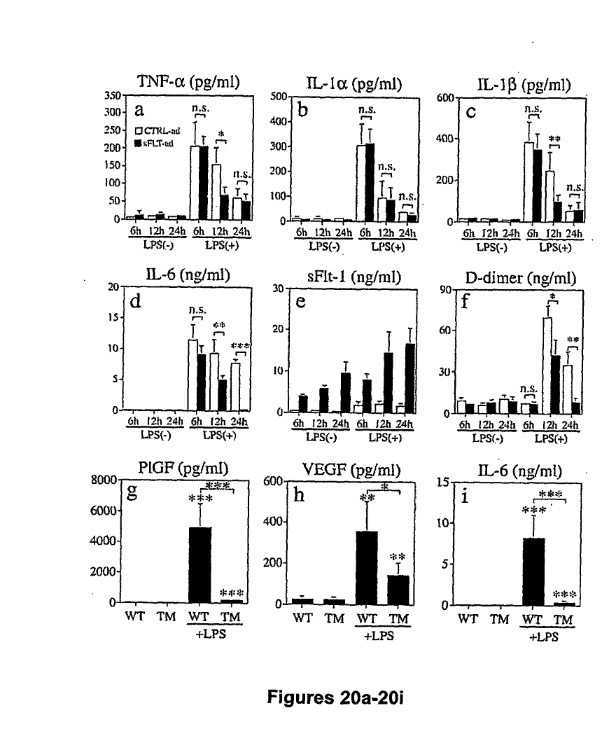

Exogenous sFlt-1 had no effect on the initial 6 h peak of plasma cytokines induced during endotoxemia (FIGS. 20a-20i). However, cytokine levels fell more rapidly in animals that had received Ad-sFlt-1 compared to control adenovirus. Ad-sFlt-1 significantly blunted LPS-mediated induction of TNF-α, IL-1β, and IL-6, and D-dimers at 12 h and/or 24 h (FIGS. 20a-20f). LPS administration in triple mutant mice null for IL-1RI and the two TNF-α receptors, TNFR1 and TNFR2, resulted in significantly lower induction of VEGF and PlGF compared to wild type controls at 24 h (FIGS. 20g-20i). Together, these findings suggest that VEGF and PlGF lie downstream of commonly implicated sepsis-induced cytokines.

These results indicate that sFlt-1, VEGF, and PlGF are novel diagnostic, screening, and therapeutic targets in treating a subject with an inflammatory response such as sepsis, severe sepsis, or septic shock.

### Example 2

### Diagnosing an Inflammatory Response

The present invention provides assays useful in the diagnosis of an inflammatory response such as sepsis, severe sepsis, and septic shock, based on the discovery that serum levels of VEGF, PlGF, and sFlt-1 are increased in an inflammatory response. Accordingly, diagnosis of inflammatory response can be performed by measuring the level of expression or activity of VEGF, PlGF, and sFlt-1 in a sample taken from a subject. This level of expression or activity can then be compared to a control sample, for example, a sample taken from a control subject, and an increase in VEGF, PlGF, or sFlt-1 relative to the control is taken as diagnostic of an inflammatory response, or being at risk of or having a propensity to develop an inflammatory response.

Analysis of levels of VEGF, PlGF, or sFlt-1 polypeptides, or activity of the polypeptides, may be used as the basis for screening the subject sample, e.g., a sample comprising blood or urine. Methods for screening polypeptide levels may include immunological techniques standard in the art (e.g., Western blot or ELISA) using antibodies that specifically bind VEGF, PlGF, or sFlt-1 or may be performed using chromatographic or other protein purification techniques. In another embodiment, the activity of VEGF, PlGF, or sFlt-1 may be measured, where an increase in activity relative to sample taken from a control subject is diagnostic of the inflammatory response. VEGF activity may be measured, for example, as described in U.S. Pat. No. 6,787,323.

Analysis of levels VEGF, PlGF, or sFlt-1 polynucleotides may also be used as a basis for screening a subject sample. In one embodiment, mRNA levels in a sample taken from a subject are analyzed and an increase in mRNA levels compared to a control sample (e.g., a sample taken from a control subject) is indicative of an inflammatory response, or a propensity for developing an inflammatory response. Methods for screening mRNA levels include any of those standard in the art, for example, Northern blotting. mRNA levels may also be analyzed using PCR techniques such as quantitative reverse transcriptase (RT)-PCR, a method standard in the art.

Analysis VEGF, PlGF, or sFlt-1 polynucleotides may also performed on the corresponding genomic sequences to identify subjects having a genetic variation, mutation, or polymorphism in an sFlt-1, PlGF, or VEGF polynucleotide that are indicative of a predisposition to develop an inflammatory condition. Such polymorphisms are known in the art and are described by Parry et al. (Eur. J Immunogenet. 26:321-3, 1999). Such genetic alterations may be present in the promoter sequence, an open reading frame, intronic sequence, or untranslated 3′ region of an sFlt-1 gene. Information related to genetic alterations can be used to diagnose a subject as having an inflammatory response or a propensity to develop such conditions. As noted herein, specific alterations in the levels of biological activity of sFlt-1, VEGF, and/or PlGF can be correlated with the likelihood of developing inflammation, or the predisposition to the same. As a result, one skilled in the art, having detected a given mutation, can then assay biological activity of the protein to determine if the mutation causes or increases the likelihood of developing an inflammatory response.

Analysis of polynucleotides in the above-described methods may be carried out by direct analysis of the sequence of an sFlt-1, VEGF, or PlGF nucleic acid molecule. For example, direct analysis may be used to diagnose humans for a propensity to develop an inflammatory response.

The diagnostic methods described herein can be used individually or in combination with any other diagnostic method described herein for a more accurate diagnosis of the presence of, severity of, or estimated time of onset of an inflammatory response (e.g., sepsis, severe sepsis, or septic shock). In addition, the diagnostic methods described herein can be used in combination with any other diagnostic methods (e.g., measurements of IL6 or TNF-α expression or activity, or a D-dimer assay) determined to be useful for the accurate diagnosis of the presence of, severity of, or estimated time of onset of an inflammatory response.

### Diagnostic Kits

The invention also provides for a diagnostic test kit. For example, a diagnostic test kit can include antibodies to VEGF, PlGF, or sFlt-1, and means for detecting, and for evaluating, binding between the antibodies and the VEGF, PlGF, or sFlt-1 polypeptide. For detection, either the antibody or the VEGF, PlGF, or sFlt-1 polypeptide is labeled, and either the antibody or the VEGF, PlGF, or sFlt-1 polypeptide is substrate-bound, such that the VEGF, PlGF, or sFlt-1 polypeptide-antibody interaction can be established by determining the amount of label attached to the substrate following binding between the antibody and the VEGF, PlGF, or sFlt-1 polypeptide. A conventional ELISA is a common, method known in the art for detecting antibody-substrate interaction and can be provided with the kit of the invention. VEGF, PlGF, or sFlt-1 polypeptides can be detected in virtually any bodily fluid including, but not limited to urine, serum, plasma, saliva, amniotic fluid, or cerebrospinal fluid. A kit that determines an alteration in the level of VEGF, PlGF, or sFlt-1 polypeptide relative to a reference, such as the level present in a normal control, is useful as a diagnostic kit in the methods of the invention.

### Example 3

### Screening Methods for Identification of Candidate Compounds

The invention also provides screening methods for the identification of compounds that bind to, or modulate expression or activity of VEGF, PlGF, or sFlt-1, that may be useful in the treatment of an inflammatory response such as sepsis, severe sepsis, or septic shock Useful compounds decrease the expression or activity of VEGF, increase or decrease the expression or activity of PlGF, or increase the expression or activity of sFlt-1.

### Screening Assays

Screening assays to identify compounds that decrease VEGF expression or activity, increase or decrease PlGF expression or activity, or increase sFlt-1 expression or activity are carried out by standard methods. The screening methods may involve high-throughput techniques. In addition, these screening techniques may be carried out in cultured cells or in non-human organisms. Screening in these organisms may include the use of polynucleotides homologous to human VEGF, PlGF, or sFlt-1. For example, a screen in mice may include measuring the effect of candidate compounds on expression or activity of the mouse Vegfa or Pgf gene.

Any number of methods is available for carrying out such screening assays. According to one approach, candidate compounds are added at varying concentrations to the culture medium of cells expressing a polynucleotide coding for VEGF, PlGF, or sFlt-1. Gene expression is then measured, for example, by standard Northern blot analysis (Ausubel et al., Current Protocols in Molecular Biology, Wiley Interscience, New York, 1997), using any appropriate fragment prepared from the polynucleotide molecule as a hybridization probe. The level of gene expression in the presence of the candidate compound is compared to the level measured in a control culture medium lacking the candidate molecule. A compound which promotes an increase in sFlt-1 expression or a decrease in VEGF or PlGF expression is considered useful in the invention; such a molecule may be used, for example, as a therapeutic for an inflammatory response (e.g., sepsis, severe sepsis, or septic shock).

If desired, the effect of candidate compounds may, in the alternative, be measured at the level of polypeptide production using the same general approach and standard immunological techniques, such as ELISA, Western blotting or immunoprecipitation with an antibody specific for VEGF, PlGF, or sFlt-1. For example, immunoassays may be used to detect or monitor the expression of VEGF, PlGF, or sFlt-1. Polyclonal or monoclonal antibodies which are capable of binding to such a polypeptide may be used in any standard immunoassay format (e.g., ELISA, Western blot, or RIA assay) to measure the level of VEGF, PlGF, or sFlt-1. A compound which promotes an increase the expression of sFlt-1, an increase or decrease in the expression of PlGF, or a decrease in the expression of VEGF is considered particularly useful. Again, such a molecule may be used, for example, as a therapeutic for an inflammatory response (e.g., sepsis, severe sepsis, or septic shock).

Alternatively, or in addition, candidate compounds may be screened for those which specifically bind to and activate sFlt-1 or PlGF, or inhibit VEGF or PlGF. The efficacy of such a candidate compound is dependent upon its ability to interact with the polypeptide. Such an interaction can be readily assayed using any number of standard binding techniques and functional assays (e.g., those described in Ausubel et al., supra). For example, a candidate compound may be tested in vitro for interaction and binding with VEGF, PlGF, or sFlt-1 and its ability to modulate its activity may be assayed by any standard assays (e.g., those described herein).

In one particular embodiment, a candidate compound that binds to VEGF, PlGF, or sFlt-1 may be identified using a chromatography-based technique. For example, recombinant VEGF, PlGF, or sFlt-1 may be purified by standard techniques from cells engineered to express VEGF, PlGF, or sFlt-1 and may be immobilized on a column. A solution of candidate compounds is then passed through the column, and a compound specific for VEGF, PlGF, or sFlt-1 is identified on the basis of its ability to bind to the polypeptide and be immobilized on the column. To isolate the compound, the column is washed to remove non-specifically bound molecules, and the compound of interest is then released from the column and collected. Compounds isolated by this method (or any other appropriate method) may, if desired, be further purified (e.g., by high performance liquid chromatography). Compounds isolated by this approach may also be used, for example, as therapeutics to treat an inflammatory response (e.g., sepsis, severe sepsis, or septic shock). Compounds which are identified as binding to VEGF, PlGF, or sFlt-1 with an affinity constant less than or equal to 10 mM are considered particularly useful in the invention.

Potential agonists and antagonists include organic molecules, peptides, peptide mimetics, polypeptides, and antibodies that bind to VEGF, PlGF, or sFlt-1, or a polynucleotide encoding VEGF, PlGF, or sFlt-1 and thereby increase or decrease its activity. Potential antagonists include small molecules that bind to VEGF or PlGF and prevent these proteins from binding their receptors. Other potential antagonists include antisense molecules. Alternatively, small molecules may act as agonists and bind sFlt-1 such that its activity is increased.

Polynucleotide sequences coding for VEGF, PlGF, or sFlt-1 may also be used in the discovery and development of compounds to treat an inflammatory response (e.g., sepsis, severe sepsis, or septic shock). VEGF, PlGF, or sFlt-1, upon expression, can be used as a target for the screening of drugs. Additionally, the polynucleotide sequences encoding the amino terminal regions of the encoded polypeptide or Shine-Delgarno or other translation facilitating sequences of the respective mRNA can be used to construct antisense sequences to control the expression of the coding sequence of interest. Polynucleotides encoding fragments of VEGF or PlGF may, for example, be expressed such that RNA interference takes place, thereby reducing expression or activity of VEGF or PlGF.

The antagonists and agonists of the invention may be employed, for instance, to treat a variety of inflammatory responses such as sepsis, severe sepsis, and septic shock.

Optionally, compounds identified in any of the above-described assays may be confirmed as useful in delaying or ameliorating an inflammatory response in either standard tissue culture methods or animal models and, if successful, may be used as therapeutics for treating an inflammatory response.

Small molecules provide useful candidate therapeutics. Such molecules may have a molecular weight below 2,000 daltons, between 300 and 1,000 daltons, or between 400 and 700 daltons. These small molecules may be organic molecules.

### Test Compounds and Extracts

In general, compounds capable of treating an inflammatory response (e.g., sepsis, severe sepsis, or septic shock) are identified from large libraries of both natural product or synthetic (or semi-synthetic) extracts or chemical libraries according to methods known in the art. Those skilled in the field of drug discovery and development will understand that the precise source of test extracts or compounds is not critical to the screening procedure(s) of the invention. Accordingly, virtually any number of chemical extracts or compounds can be screened using the methods described herein. Examples of such extracts or compounds include, but are not limited to, plant-, fungal-, prokaryotic- or animal-based extracts, fermentation broths, and synthetic compounds, as well as modification of existing compounds. Numerous methods are also available for generating random or directed synthesis (e.g., semi-synthesis or total synthesis) of any number of chemical compounds, including, but not limited to, saccharide-, lipid-, peptide-, and polynucleotide-based compounds. Synthetic compound libraries are commercially available. Alternatively, libraries of natural compounds in the form of bacterial, fungal, plant, and animal extracts are commercially available. In addition, natural and synthetically produced libraries are produced, if desired, according to methods known in the art, e.g., by standard extraction and fractionation methods. Furthermore, if desired, any library or compound is readily modified using standard chemical, physical, or biochemical methods.

In addition, those skilled in the art of drug discovery and development readily understand that methods for dereplication (e.g., taxonomic dereplication, biological dereplication, and chemical dereplication, or any combination thereof) or the elimination of replicates or repeats of materials already known for their activity in treating inflammatory responses should be employed whenever possible.

When a crude extract is found to have an activity that increases sFlt-1 or PlGF expression or activity or decreases VEGF or PlGF expression or activity, or a binding activity, further fractionation of the positive lead extract is necessary to isolate chemical constituents responsible for the observed effect. Thus, the goal of the extraction, fractionation, and purification process is the characterization and identification of a chemical entity within the crude extract having activity that may be useful in treating an inflammatory response (e.g., sepsis, severe sepsis, or septic shock). Methods of fractionation and purification of such heterogenous extracts are known in the art. If desired, compounds shown to be useful agents for the treatment of an inflammatory response (e.g., sepsis, severe sepsis, or septic shock) are chemically modified according to methods known in the art.

### Example 4

### Treating an Inflammatory Response

The invention also provides methods of treating a subject with an inflammatory response such as sepsis, severe sepsis, or septic shock. Treatment methods include administration of compounds that increase the amount or activity of sFlt-1 or PlGF or reduce the amount or activity of VEGF or PlGF in a subject with an inflammatory response (e.g., sepsis, severe sepsis, or septic shock).

sFlt-1 and PlGF Treatment of a subject with an inflammatory response such as sepsis, severe sepsis, or septic shock may be achieved by administration of sFlt-1 or PlGF. Administration may be by any route described herein; however, parenteral administration is preferred and intravenous administration is more preferred. Administration may be amounts such that concentrations of sFlt-1 are increased by 2-50 fold, e.g. 5-10 fold over basal (i.e., untreated) levels of Flt. Likewise, administration of PlGF may increase levels of PlGF over basal (i.e., untreated) by 1.1-200 fold over basal, e.g., 10-100 fold. Additionally, the sFlt1-1 or PlGF polypeptide administered may include modifications such as post-translational modifications (e.g., glycosylation, phosphorylation), or other chemical modifications, for example, modifications designed to alter distribution of sFlt-1 or PlGF within the subject or alter rates of degradation and/or excretion of sFlt-1 or PlGF.

### Anti-VEGF and Anti-PlGF Antibodies

Treatment of a subject with an inflammatory response may also be achieved by administration of anti-VEGF or anti-PlGF antibodies (for example, monoclonal antibodies) that specifically bind the VEGF or PlGF protein. Anti-VEGF antibodies, for example as described in U.S. Pat. No. 6,884,879, may be used in the treatment methods of the invention. Other useful anti-VEGF antibodies include bevacizumab (Avastin, Genentech, South San Francisco, Calif.). VEGF and PlGF antibodies are also available from R&D Systems, Minneapolis, Minn. Other VEGF antibodies include HuMV833, 2C3 (Peregrine Pharmaceuticals, Tustin, Calif.), and VEGF-trap (Regeneron Pharmaceuticals, Inc., Tarrytown, N.Y.).

Additionally, antibodies may be made by any standard method and tested for their ability to block VEGF or PlGF activity either directly or indirectly. These antibodies may be modified in any way to make them more appropriate for human administration. For example, they may be single-chain antibodies or humanized antibodies. Again, these antibodies are administered by any route, formulation, frequency, or in any dose that achieves in vivo concentrations sufficient for treatment of an inflammatory response.

### VEGF- and PlGF-Receptor Inhibitory Compounds

Another approach to treating an inflammatory response (e.g., sepsis, severe sepsis, and septic shock) may be by treating a subject with an inhibitory compound (e.g., an antibody) specifically binds a receptor for VEGF or PlGF. VEGF receptors include VEGFR-1 (Flt-1) VEGFR-2 (Flk-1/KDR), VEGFR-3, and neuropilin-1; PlGF receptors include, VEGFR-1 (Flt-1), and neuropilin-1. Such antibodies are known in the art (e.g., antibodies that specifically bind Flk-1 and Flt-1 (R&D Systems); 2C7, a humanized anti-VEGFR-2 monoclonal antibody), or may be generated used methods standard in the art and tested for their ability to bind VEGF or PlGF receptors (e.g., those described herein) either directly or indirectly. These antibodies may be modified in any way to make them more appropriate for human administration. For example, they may be single-chain antibodies or humanized antibodies. Again, these antibodies are administered by any route, formulation, frequency, or in any dose that achieves in vivo concentrations sufficient for treatment of an inflammatory response. As described above, antibodies for treatment may be generated by methods standard in the art as well.

Other inhibitory compounds include SU5416 (semaxanib), SU6668, SU011248, PTK-787/ZK222584, ZD6474, CD-547632, AG-013736, and CEP-7055 (Bergsland, E. K. Am J Health-Syst Pharm 61(Suppl 5), S4-S11; Bergsland, E. K., Am J Health-Syst Pharm 61 (Suppl 5), S12-S20).

### VEGF Inhibitory Compounds

Any compounds that inhibit VEGF or VEGF activity are also useful in the treatment methods of the invention. Pyridine derivatives and analogs which inhibit VEGF are described in, for example, U.S. Pat. No. 6,706,731. Oligonucleotides with high affinity binding to VEGF are described, for example in U.S. Pat. No. 6,696,252. 2-Amino-nicotinamide derivatives that act as VEGF-receptor inhibitors are described, for example, in U.S. Pat. Nos. 6,624,174 and 6,878,714. Peptides that inhibit VEGF activity are described in U.S. Pat. Nos. 6,559,126, 5,861,484, 6,383,486, 6,100,071, 6,270,993, 6,777,534.

### Gene Therapy/Therapeutic Nucleic Acids

Increases in sFlt-1 or PlGF expression or activity or decreases in VEGF or PlGF expression or activity may also be achieved through introduction of gene vectors into a subject. Recent work has shown that the delivery of nucleic acid (DNA or RNA) capable of expressing an endothelial cell mitogen such as VEGF to the site of a blood vessel injury will induce proliferation and reendothelialization of the injured vessel. While the present invention does not relate to blood vessel injury and seeks to reduce VEGF levels, the techniques for the delivery of nucleic acid encoding endothelial cell mitogens such as sFlt-1 and PlGF used in these studies can also be employed in the present invention. These techniques are described in U.S. Pat. Nos. 5,830,879 and 6,258,787 and are incorporated herein by reference.

In the present invention the nucleic acid may be any nucleic acid (DNA or RNA) including genomic DNA, cDNA, and mRNA, encoding sFlt-1, VEGF, or PlGF or any sFlt-1, VEGF, or PlGF family members. The nucleic acid may also include any nucleic acid which encodes a protein shown to bind to a VEGF or PlGF receptor. The nucleic acids encoding the desired protein may be obtained using routine procedures in the art, e.g., recombinant DNA, PCR amplification.

To treat an inflammatory response such as sepsis, severe sepsis, or septic shock, sFlt-1 or PlGF expression may be increased, for example, by administering to a subject a vector containing a polynucleotide sequence encoding sFlt-1 or PlGF, operably linked to a promoter capable of driving expression in targeted cells. In another approach, a polynucleotide sequence encoding a protein that increases transcription of the sFlt-1 or PlGF gene may be administered to a subject with an inflammatory response. Any standard gene therapy vector and methodology may be employed for such administration.

Alternatively, to decrease expression of VEGF or PlGF for treating an inflammatory response such as sepsis, severe sepsis, or septic shock, RNA interference (RNAi) may be employed. Vectors containing a target sequence, such as a short (for example, 19 base pair) sense target sequence and corresponding antisense target sequence joined by a short (for example, 9 base pair) sequence capable of forming a stem-loop structure, of the VEGF or PlGF mRNA transcript may be administered to a subject with an inflammatory response. In one embodiment, the ribozyme, angiozyme, is administered to patients with an inflammatory response. When this vector is expressed in cells, small, inhibitory RNA (siRNA) molecules are generated from this stem-loop structure, and these bind to VEGF or PlGF mRNA transcripts, which results in increased degradation of the targeted mRNA transcripts relative to untargeted transcripts. The use of antisense nucleobase oligomers to downregulate VEGF expression is described in U.S. Pat. No. 6,410,322, incorporated herein by reference. To test the efficacy of different sequences in mammalian cell culture systems, the pSuper RNAi System (OligoEngine, Seattle, Wash.), for example, may be employed.

### Combination Therapies

Combination therapies may be performed by 1) combining two or more of the treatment methods described herein (e.g., treating with a compound and a nucleic acid, where the compound inhibits VEGF and the nucleic acid increases sFlt-1 expression) or 2) using the treatment methods described herein along with previously existing treatments for inflammatory responses are also provided by the invention. Previously existing treatment methods include administration of antimicrobials (e.g., broad-spectrum antibiotics such as penicillin, ampicillin, bacitracin, carbapenems, cephalosporin, methicillin, oxacillin, vancomycin), fluids, vasopressors, corticosteroids, activated protein C (Xigris, Eli Lily and Co.), and glucose and insulin administration; mechanical ventilation; dialysis; and sedation.

### Formulation of Pharmaceutical Compositions

The administration of any compound described herein (e.g., VEGF antibodies and sFlt-1) or identified using the methods of the invention may be by any suitable means that results in a concentration of the compound that treats a an inflammatory response. The compound may be contained in any appropriate amount in any suitable carrier substance, and is generally present in an amount of 1-95% by weight of the total weight of the composition. The composition may be provided in a dosage form that is suitable for the oral, parenteral (e.g., intravenously or intramuscularly), rectal, cutaneous, nasal, vaginal, inhalant, skin (patch), ocular, or intracranial administration route. Thus, the composition may be in the form of, e.g., tablets, capsules, pills, powders, granulates, suspensions, emulsions, solutions, gels including hydrogels, pastes, ointments, creams, plasters, drenches, osmotic delivery devices, suppositories, enemas, injectables, implants, sprays, or aerosols. The pharmaceutical compositions may be formulated according to conventional pharmaceutical practice (see, e.g., Remington: The Science and Practice of Pharmacy, 20th edition, 2000, ed. A. R. Gennaro, Lippincott Williams & Wilkins, Philadelphia, and Encyclopedia of Pharmaceutical Technology, eds. J. Swarbrick and J. C. Boylan, 1988-1999, Marcel Dekker, New York).

Pharmaceutical compositions may be formulated to release the active compound immediately upon administration or at any predetermined time or time period after administration. The latter types of compositions are generally known as controlled release formulations, which include (i) formulations that create substantially constant concentrations of the agent(s) of the invention within the body over an extended period of time; (ii) formulations that after a predetermined lag time create substantially constant concentrations of the agents of the invention within the body over an extended period of time; (iii) formulations that sustain the agent(s) action during a predetermined time period by maintaining a relatively constant, effective level of the agent(s) in the body with concomitant minimization of undesirable side effects associated with fluctuations in the plasma level of the agent(s) (sawtooth kinetic pattern); (iv) formulations that localize action of agent(s), e.g., spatial placement of a controlled release composition adjacent to or in the diseased tissue or organ; (v) formulations that achieve convenience of dosing, e.g., administering the composition once per week or once every two weeks; and (vi) formulations that target the action of the agent(s) by using carriers or chemical derivatives to deliver the compound to a particular target cell type. Administration of the compound in the form of a controlled release formulation is especially preferred for compounds having a narrow absorption window in the gastro-intestinal tract or a relatively short biological half-life.

Any of a number of strategies can be pursued in order to obtain controlled release in which the rate of release outweighs the rate of metabolism of the compound in question. In one example, controlled release is obtained by appropriate selection of various formulation parameters and ingredients, including, e.g., various types of controlled release compositions and coatings. Thus, the compound is formulated with appropriate excipients into a pharmaceutical composition that, upon administration, releases the compound in a controlled manner. Examples include single or multiple unit tablet or capsule compositions, oil solutions, suspensions, emulsions, microcapsules, molecular complexes, microspheres, nanoparticles, patches, and liposomes.

### Parenteral Compositions

The composition containing compounds described herein or identified using the methods of the invention may be administered parenterally by injection, infusion, or implantation (subcutaneous, intravenous, intramuscular, intraperitoneal, or the like) in dosage forms, formulations, or via suitable delivery devices or implants containing conventional, non-toxic pharmaceutically acceptable carriers and adjuvants. The formulation and preparation of such compositions are well known to those skilled in the art of pharmaceutical formulation.

Compositions for parenteral use may be provided in unit dosage forms (e.g., in single-dose ampoules), or in vials containing several doses and in which a suitable preservative may be added (see below). The composition may be in form of a solution, a suspension, an emulsion, an infusion device, or a delivery device for implantation, or it may be presented as a dry powder to be reconstituted with water or another suitable vehicle before use. Apart from the active agent(s), the composition may include suitable parenterally acceptable carriers and/or excipients. The active agent(s) may be incorporated into microspheres, microcapsules, nanoparticles, liposomes, or the like for controlled release. Furthermore, the composition may include suspending, solubilizing, stabilizing, pH-adjusting agents, tonicity adjusting agents, and/or dispersing agents.

As indicated above, the pharmaceutical compositions according to the invention may be in a form suitable for sterile injection. To prepare such a composition, the suitable active agent(s) are dissolved or suspended in a parenterally acceptable liquid vehicle. Among acceptable vehicles and solvents that may be employed are water, water adjusted to a suitable pH by addition of an appropriate amount of hydrochloric acid, sodium hydroxide or a suitable buffer, 1,3-butanediol, Ringer's solution, dextrose solution, and isotonic sodium chloride solution. The aqueous formulation may also contain one or more preservatives (e.g., methyl, ethyl, or n-propyl p-hydroxybenzoate). In cases where one of the compounds is only sparingly or slightly soluble in water, a dissolution enhancing or solubilizing agent can be added, or the solvent may include 10-60% w/w of propylene glycol or the like.

### Controlled Release Parenteral Compositions

Controlled release parenteral compositions may be in the form of aqueous suspensions, microspheres, microcapsules, magnetic microspheres, oil solutions, oil suspensions, or emulsions. The composition may also be incorporated in biocompatible carriers, liposomes, nanoparticles, implants, or infusion devices.

Materials for use in the preparation of microspheres and/or microcapsules are, e.g., biodegradable/bioerodible polymers such as polygalactin, poly-(isobutyl cyanoacrylate), poly(2-hydroxyethyl-L-glutamine), poly(lactic acid), polyglycolic acid, and mixtures thereof. Biocompatible carriers that may be used when formulating a controlled release parenteral formulation are carbohydrates (e.g., dextrans), proteins (e.g., albumin), lipoproteins, or antibodies. Materials for use in implants can be non-biodegradable (e.g., polydimethyl siloxane) or biodegradable (e.g., poly(caprolactone), poly(lactic acid), poly(glycolic acid) or poly(ortho esters)) or combinations thereof.

### Solid Dosage Forms for Oral Use

Formulations for oral use include tablets containing the active ingredient(s) in a mixture with non-toxic pharmaceutically acceptable excipients, and such formulations are known to the skilled artisan (e.g., U.S. Pat. Nos. 5,817,307, 5,824,300, 5,830,456, 5,846,526, 5,882,640, 5,910,304, 6,036,949, 6,036,949, 6,372,218, hereby incorporated by reference). These excipients may be, for example, inert diluents or fillers (e.g., sucrose, sorbitol, sugar, mannitol, microcrystalline cellulose, starches including potato starch, calcium carbonate, sodium chloride, lactose, calcium phosphate, calcium sulfate, or sodium phosphate); granulating and disintegrating agents (e.g., cellulose derivatives including microcrystalline cellulose, starches including potato starch, croscarmellose sodium, alginates, or alginic acid); binding agents (e.g., sucrose, glucose, sorbitol, acacia, alginic acid, sodium alginate, gelatin, starch, pregelatinized starch, microcrystalline cellulose, magnesium aluminum silicate, carboxymethylcellulose sodium, methylcellulose, hydroxypropyl methylcellulose, ethylcellulose, polyvinylpyrrolidone, or polyethylene glycol); and lubricating agents, glidants, and anti-adhesives (e.g., magnesium stearate, zinc stearate, stearic acid, silicas, hydrogenated vegetable oils, or talc). Other pharmaceutically acceptable excipients can be colorants, flavoring agents, plasticizers, humectants, buffering agents, and the like.

The tablets may be uncoated or they may be coated by known techniques, optionally to delay disintegration and absorption in the gastrointestinal tract and thereby providing a sustained action over a longer period. The coating may be adapted to release the compound in a predetermined pattern (e.g., in order to achieve a controlled release formulation) or it may be adapted not to release the agent(s) until after passage of the stomach (enteric coating). The coating may be a sugar coating, a film coating (e.g., based on hydroxypropyl methylcellulose, methylcellulose, methyl hydroxyethylcellulose, hydroxypropylcellulose, carboxymethylcellulose, acrylate copolymers, polyethylene glycols, and/or polyvinylpyrrolidone), or an enteric coating (e.g., based on methacrylic acid copolymer, cellulose acetate phthalate, hydroxypropyl methylcellulose phthalate, hydroxypropyl methylcellulose acetate succinate, polyvinyl acetate phthalate, shellac, and/or ethylcellulose). Furthermore, a time delay material such as, e.g., glyceryl monostearate or glyceryl distearate, may be employed.

The solid tablet compositions may include a coating adapted to protect the composition from unwanted chemical changes, (e.g., chemical degradation prior to the release of the active substances). The coating may be applied on the solid dosage form in a similar manner as that described in Encyclopedia of Pharmaceutical Technology, supra.

The compositions of the invention may be mixed together in the tablet, or may be partitioned. In one example, a first agent is contained on the inside of the tablet, and a second agent is on the outside, such that a substantial portion of the second agent is released prior to the release of the first agent.

Formulations for oral use may also be presented as chewable tablets, or as hard gelatin capsules wherein the active ingredient is mixed with an inert solid diluent (e.g., potato starch, lactose, microcrystalline cellulose, calcium carbonate, calcium phosphate, or kaolin), or as soft gelatin capsules wherein the active ingredient is mixed with water or an oil medium, for example, peanut oil, liquid paraffin, or olive oil. Powders and granulates may be prepared using the ingredients mentioned above under tablets and capsules in a conventional manner using, e.g., a mixer, a fluid bed apparatus, or spray drying equipment.

### Controlled Release Oral Dosage Forms

Controlled release compositions for oral use may, e.g., be constructed to release the compound by controlling the dissolution and/or the diffusion of the compound.

Dissolution or diffusion controlled release can be achieved by appropriate coating of a tablet, capsule, pellet, or granulate formulation of compounds, or by incorporating the compound into an appropriate matrix. A controlled release coating may include one or more of the coating substances mentioned above and/or, e.g., shellac, beeswax, glycowax, castor wax, carnauba wax, stearyl alcohol, glyceryl monostearate, glyceryl distearate, glycerol palmitostearate, ethylcellulose, acrylic resins, DL-polylactic acid, cellulose acetate butyrate, polyvinyl chloride, polyvinyl acetate, vinyl pyrrolidone, polyethylene, polymethacrylate, methylmethacrylate, 2-hydroxymethacrylate, methacrylate hydrogels, 1,3 butylene glycol, ethylene glycol methacrylate, and/or polyethylene glycols. In a controlled release matrix formulation, the matrix material may also include, e.g., hydrated methylcellulose, carnauba wax, and stearyl alcohol, carbopol 934, silicone, glyceryl tristearate, methyl acrylate-methyl methacrylate, polyvinyl chloride, polyethylene, and/or halogenated fluorocarbon.

A controlled release composition containing compounds described herein or identified using methods of the invention may also be in the form of a buoyant tablet or capsule (i.e., a tablet or capsule that, upon oral administration, floats on top of the gastric content for a certain period of time). A buoyant tablet formulation of the compound(s) can be prepared by granulating a mixture of the composition with excipients and 20-75% w/w of hydrocolloids, such as hydroxyethylcellulose, hydroxypropylcellulose, or hydroxypropylmethylcellulose. The obtained granules can then be compressed into tablets. On contact with the gastric juice, the tablet forms a substantially water-impermeable gel barrier around its surface. This gel barrier takes part in maintaining a density of less than one, thereby allowing the tablet to remain buoyant in the gastric juice.

### Dosages

The dosage of any compound described herein or identified using the methods described herein depends on several factors, including: the administration method, the inflammatory response to be treated, the severity of the inflammatory response, whether the inflammatory response is to be treated or prevented, and the age, weight, and health of the subject to be treated.

With respect to the treatment methods of the invention, it is not intended that the administration of a compound to a subject be limited to a particular mode of administration, dosage, or frequency of dosing; the present invention contemplates all modes of administration, including intramuscular, intravenous, intraperitoneal, intravesicular, intraarticular, intralesional, subcutaneous, or any other route sufficient to provide a dose adequate to treat hepatitis. The compound may be administered to the subject in a single dose or in multiple doses. For example, a compound described herein or identified using screening methods of the invention may be administered once a week for, e.g., 2, 3, 4, 5, 6, 7, 8, 10, 15, 20, or more weeks. It is to be understood that, for any particular subject, specific dosage regimes should be adjusted over time according to the individual need and the professional judgment of the person administering or supervising the administration of the compound. For example, the dosage of a compound can be increased if the lower dose does not provide sufficient activity in the treatment of an inflammatory response (e.g., sepsis, severe sepsis, or septic shock). Conversely, the dosage of the compound can be decreased if the inflammatory response is reduced or eliminated.

While the attending physician ultimately will decide the appropriate amount and dosage regimen, a therapeutically effective amount of a compound described herein (e.g., VEGF antibodies or sFlt-1) or identified using the screening methods of the invention, may be, for example, in the range of 0.0035 μg to 20 μg/kg body weight/day or 0.010 μg to 140 μg/kg body weight/week. Desirably a therapeutically effective amount is in the range of 0.025 μg to 10 μg/kg, for example, at least 0.025, 0.035, 0.05, 0.075, 0.1, 0.25, 0.5, 1.0, 1.5, 2.0, 2.5, 3.0, 3.5, 4.0, 5.0, 6.0, 7.0, 8.0, or 9.0 μg/kg body weight administered daily, every other day, or twice a week. In addition, a therapeutically effective amount may be in the range of 0.05 μg to 20 μg/kg, for example, at least 0.05, 0.7, 0.15, 0.2, 1.0, 2.0, 3.0, 4.0, 5.0, 6.0, 7.0, 8.0, 10.0, 12.0, 14.0, 16.0, or 18.0 μg/kg body weight administered weekly, every other week, or once a month. Furthermore, a therapeutically effective amount of a compound may be, for example, in the range of 100 μg/m² to 100,000 μg/m² administered every other day, once weekly, or every other week. In a desirable embodiment, the therapeutically effective amount is in the range of 100 μg/m² to 20,000 μg/m², for example, at least 1000, 1500, 4000, or 14,000 μg/m² of the compound administered daily, every other day, twice weekly, weekly, or every other week.

### Methods and Materials

The following methods and materials were used to carry out the experiments described above.

Mouse models of endotoxemia, CLP, and pneumonia. LPS injections, CLP, and pneumonia models were carried out in male 8-week-old C57BL/6 (22-24 gram body weight). For the endotoxemia model, mice were intraperitoneally (IP) injected with normal saline (control) or LPS (18 mg/kg weight) from Escherichia coli serotype 0111:B4 (Sigma, St. Louis, Mo.). Where indicated, LPS was administered to triple mutant (TM) mice null for TNF receptor 1 (TNFR1), TNFR2, and the type I IL-1 receptor (IL1RI), or wild type (WT) controls on matched C57BL/6×129/Sv background. TM mice were maintained in a full barrier facility under specific pathogen free conditions (Mizgerd et al., Am J Physiol Lung Cell Mol Physiol 286:L1302-1310). CLP was performed as previously described with minor modifications (Rice et al., J Infect Dis 191:1368-1376, 2005). Briefly, mice were anesthetized with isofluorane. After shaving the abdomen, a 2-cm midline incision was created under aseptic conditions to expose the cecum and adjoining intestine. Approximately 25-30% of the cecum was ligated distal to the ileo-cecal valve with a 4-0 vicryl suture, and then punctured with a 21-gauge needle. The cecum was then gently squeezed to extrude a small amount of feces to ensure patency of the perforation sites and returned to the peritoneal cavity. Mice received 1 ml of saline IP for fluid resuscitation at the time of closure and 0.1 mg/kg buprenorphine SC every 12 hr to minimize distress. For the pneumonia model, mice were anesthetized by intramuscular (IM) injection of ketamine hydrochloride (100 mg/kg), acepromazine maleate (5 mg/kg IM), and atropine (0.1 mg/kg IM). The trachea was surgically exposed, and Escherichia coli (strain 19138 from the American Type Culture Collection; Manassas, Va.) were intratracheally instilled at 10⁶ CFU/mouse via angiocatheter through the trachea to the left bronchus. After 6 or 24 h, mice were sacrificed by halothane inhalation.

Human models of endotoxeinia and sepsis. Briefly, the subjects underwent comprehensive screening and were included if they had no current or past history of psychiatric, neurological, immune, cardiovascular, or sleep disorders; no history of drug dependence/abuse, including cigarette smoking during the last six months; normal blood chemistry (complete and differential blood counts, T-cell subsets, glucose, creatinine, sodium, potassium, thyroid stimulating hormone) and negative blood and urine toxicology. Subjects received an intravenous injection of either placebo or a hostresponse challenge with 2 ng/kg Escherichia coli endotoxin (O:113, Lot EC-5). The human sepsis study was approved by the Research Ethics Board of the Hamilton Health Sciences (Henderson General Hospital, Hamilton, ON). Patients with severe sepsis were identified in the intensive care units of the Henderson General Hospital, St. Joseph's Healthcare, and the Hamilton General Hospital (Hamilton, Ontario, Canada) using the inclusion and exclusion criteria as previously described (Liaw et al., Blood 104:3958-3964, 2004). Patients who received Xigris were excluded from the analysis. Baseline characteristics of the patients were collected, including demographic information, acute physiology and chronic health evaluation (APACHE II) admission scores, multiple organ dysfunction (MOD) scores, co-morbidities, organ function, site and type of infection, and hematologic tests (see Table below).

| | |
|---|---|
| Age (year) Mean ± SD (min, max) | 65 ± 14 (36, 81) |
| APACHE II score Mean ± SD (min, max) | 26.6 ± 8.2 (10, 42) |
| MOD scorea Mean ± SD (min, max) | 11.1 ± 3.7 (3, 15) |
| Primary site of infection (number) | |
| Lung | 11 |
| Abdomen | 1 |
| Urinary tract | 1 |
| Other | 1 |
| Unknown | 1 |
| Positive blood cultures (number) | |
| Gram-negative bacteria | 3 |
| Gram-positive bacteria | 7 |
| Fungus | 1 |
| Mixed | 1 |
| Unknown | 3 |
| 28 day mortality rate | 47% |

^{a}Multiple organ dysfunction.

Measurement of cytokine levels in plasma, serum or tissue lysates. TNF-α and IL-1 were measured in serum. IL-6, VEGF, and PlGF were measured in plasma. To isolate plasma from mice, blood samples were collected by heart puncture into heparinized tubes, centrifuged, and the supernatant saved as plasma. To obtain mouse serum, blood samples were incubated overnight incubation at 4° C., centrifuged and the supernatant saved as serum. In the human endotoxemia study, blood samples were drawn at regular intervals in EDTA tubes and set on ice for 5 min before centrifuging at 2600g, and then plasma pipetted into aliquots and stored at −80° C. until subsequent assay. In patients with severe sepsis, blood was collected within 24 h of meeting the definition of severe sepsis. Blood was collected daily for the first 7 days and once a week thereafter for the duration of the patients' stay in the ICU. Venous blood (9 mL) was drawn via indwelling catheters from the patients. As controls, venous blood (9 mL) was drawn via venipuncture from healthy adult volunteers. For each patient or volunteer, 4.5 mL of the collected blood was immediately transferred to 15-mL polypropylene tubes containing 0.5 mL 0.105 M buffered trisodium citrate (pH 5.4), and the remaining 4.5 mL was transferred into 15-mL polypropylene tubes containing 0.5 mL 0.105 M buffered trisodium citrate (pH 5.4) and 100 μL of 1M benzamidine HCl (20 mM benzamidine final). The blood was spun at 1500 g for 10 min at 20° C., and the plasma was stored as aliquots at −80° C. To prepare mouse organ tissue lysates, mice were perfused with PBS containing 2 mM EDTA. Organs were removed and snap frozen in liquid nitrogen. Frozen tissues were homogenized in lysate buffer containing 50 mM Tris-HCl (pH 7.4), 150 mM NaCl, and 0.05% protease inhibitor cocktail (Sigma), and then centrifuged to obtain supernatant (tissue lysate). Mouse sFlt-1, VEGF, TNF-α, IL-6, IL-1α and IL-1β were assayed using Quantikine ELISA kit (R&D systems Inc). Mouse D-dimer was measured using Asserachrom DI-D, enzyme immunoassay of D-dimer (Diagnostica Stago, France). Human plasma PlGF, VEGF-A, were measured using human PlGF and VEGF Quantikine ELISA kits (R&D Systems Inc, Minneapolis, Minn.). Citrate/benzamidine plasma samples were used for APC assays using the method previously described (Liaw et al., J Thromb Haemost 1:662-670, 2003).

Ad-mediated overexpression of soluble Flt-1 (sFlt), VEGF, and PlGF. Mice were injected intravenously with Ad over-expressing GFP (control, 2×10⁸ pfu), murine sFlt-1 (1×10⁸ pfu), murine VEGF-A (isoform 120) (2×10⁸ pfu), or murine PlGF-2 (2×10⁸ pfu). The construction of these viruses has been described previously (H. A. von Recum et al., Proc Natl Acad Sci USA 98:4605-4610, 2001; Maynard et al., J Clin Invest 111:649-658, 2003; Luttun et al., Nat Med 8:831-840, 2002). The dose of Ad-sFlt-1, Ad-VEGF and Ad-PlGF was titrated to achieve plasma levels of approximately 10-25 ng/ml. Commercial Quantakine ELISA kits (R&D Systems Inc) that measure murine sFlt-1, VEGF and PlGF were used to assay circulating levels of these cytokines from mouse plasma.

Antibody administration. 16 h prior to LPS administration, mice were injected IP with injection of 800 μg anti-mouse Flk-1 antibody (DC101), 1200 jig of anti-mouse Flt-1 antibody (MF-1), or control PBS. Both antibodies were kindly provided by ImClone Systems Incorporated (New York, N.Y.).

Soluble Flt-1 peptide administration. Mice were injected intravenously with 1 μg of human recombinant soluble Flt-1 Domain D1-3 (Cell Sciences, Inc, Canton, Mass.) or equal volume PBS (control) every 3 h for 12 h beginning one hour after LPS administration or CLP.

Cardiac physiological analysis. Echocardiographic examination of mice was performed as previously described (McMullen et al., Proc Natl Acad Sci USA 100:12355-12360, 2003; Shioi et al., EMBO J. 19:2537-2548, 2000). Briefly, mice were anesthetized with IP injection of ketamine HCl (50 mg/kg) and xylazine (10 mg/kg). A Hewlett-Packard (Andover, Mass.) Sonos 1500 sector scanner equipped with a 12 MHz transducer is used to record two-dimensionally guided M-mode tracings to assess left ventricle (LV) wall thickness, LV dimensions and fractional shortening. Electrocardiogram (ECG) recordings were acquired on anesthetized mice with a multi-channel amplifier and converted to digital signals for analysis (PowerLab system; ADInstruments, Colorado Springs, Col.).

Permeability assay. Mice were anesthetized by IP injection of 0.5 ml Avertin. 100 μl of 1% Evans blue dye (in PBS) was injected into tail vein. 40 min later, mice were perfused via heart puncture with PBS containing 2 mM EDTA for 20 min. Organs (brain, heart, lung, liver, kidney, spleen) were harvested and incubated in formamide for 3 days to elute Evans blue dye. OD of formamide solution was measured using 620 nm wave length.

Tissue RNA isolation and quantitative TaqMan PCR analysis. Tissue RNA was isolated using Trizol (Invitrogen, Carlsbad, Calif.), and RNA mini preparation kit (Qiagen, Germany). For quantitative Real time PCR, total RNA was prepared using the RNeasy RNA extraction kit with DNase I treatment following the manufacturer's instructions (Qiagen, Germany). To generate cDNA, total RNA (100 ng) from each of triplicate samples was mixed and converted into cDNA using random primers and Superscript III reverse transcriptase (Invitrogen, Carlsbad, Calif.). All cDNA samples were aliquoted and stored at −80° C. Primers were designed using the Primer Express oligo design software (Applied BioSystems, Foster City, Calif.), and synthesized by Integrated DNA Technologies (Coralville, Iowa). AU primer sets were subjected to rigorous database searches to identify potential conflicting transcript matches to pseudogenes or homologous domains within related genes. Amplicons generated from the primer set were analyzed for melting point temperatures using the first derivative primer melting curve software supplied by Applied BioSystems. The SYBR Green I assay and the ABI Prism 7500 Sequence Detection System were used for detecting real-time PCR products from the reverse transcribed cDNA samples using a master template. PCR reactions for each sample were performed in duplicate and copy numbers were measured as described previously. The level of target gene expression was normalized against the 18S rRNA expression in each sample and the data presented as mRNA copies per 10⁶ 18S rRNA copies.

Immunohistochemistry. Immunohistochemistry was carried out on 5-μm frozen sections from heart, brain and lung of control and LPS-treated mice, as previously described. Antibodies included rabbit polyclonal anti-mouse P-selectin antibody (Chemicon International, Temecula, Calif.), rabbit polyclonal anti-mouse PAI-1 antibody (Innovative Research Inc, Southfield, Mich.), rabbit polyclonal anti-mouse Cox-2 antibody (Cayman Chemical, Ann Arbor, Mich.), rat monoclonal anti-mouse E-selectin antibody (BD Biosciences Pharmingen, San Diego, Calif.), rat monoclonal anti-mouse VCAM-1 antibody (Chemicon International), and rat monoclonal anti-mouse ICAM-1 antibody (Serotec Ltd, Oxford, UK). Anti-rat IgG antibody conjugated with FITC and anti-rabbit IgG antibody conjugated with Cy3 (ZYMED Laboratories, South San Francisco, Calif.) were used as secondary antibodies. For co-localization studies, a rat monoclonal anti-mouse CD31 antibody (BD Biosciences Pharmingen) was used in double immunofluorescent stains with rabbit polyclonal antibodies (P-selectin, PAI-1 and Cox-2); and a rabbit polyclonal anti-mouse vWF antibody (Abcam Inc, Cambridge, Mass.) was combined with the rat monoclonal anti-mouse antibodies (E-selectin, VCAM-1 and ICAM-1).

Cell culture. HUVEC were cultured in EGM medium (Cambrex Bio Science, Walkersville, Md.). Once cells reached 70% confluence, they were serum starved in 0.5% FBS EGM for 20 h, and then incubated with 0.1 ng/ml TNF-α, 100 ng/ml VEGF and 200 ng/ml PlGF, alone or in combination for 4 hrs. The cells were harvested for RNA and processed as described above.

Survival study. Three days before the LPS injection or CLP, C57 BL/6 male mice treated with the control (GFP)-adenovirus, sFlt-1 adenovirus, PlGF adenovirus and/or VEGF-A adenovirus. Alternatively, animals received anti-Flk-1 antibody, anti-Flt-1 antibody, or sFlt-1 peptide as described above. Survival was assessed at 24, 48, 72 and 96 h following LPS injection (20 mg/kg weight) or CLP.

In some procedures, C57BL/6 male mice (22-24 g) at eight weeks of age were injected intravenously with 1 mg of mouse PlGF-2 neutralizing antibody or control IgG at 20 hr prior to 20 mg/kg lipopolysaccharide (LPS) (SIGMA, St. Louis, Mo.) administration (i.p. injection). PlGF (−/−) male mice (FVB background) at eight weeks of age and age-matched wild-type littermates were also employed in survival studies. Mice were intravenously injected 17 mg/kg LPS. Mouse survival rate was monitored at various time points for 4 days.

Statistical analysis. Student t-test was used for statistical analysis in mouse cytokine and gene expression studies. ANOVA was used for statistical analysis of cardiac physiology data. General Linear Model (GLM) Repeated Measures were used to assess differences between human subject groups (placebo vs. endotoxin). The Greenhouse-Geisser correction of degrees of freedom was applied where appropriate. In cases of significant condition or interaction effects, simple contrasts were used to specify which time points significantly differed from each other. Survival data were analyzed by construction of Kaplan-Meier plots and use of the log-rank test.

All patents, patent applications, and publications mentioned in this specification, including U.S. provisional application 60/698,997, filed Jul. 13, 2005, are herein incorporated by reference to the same extent as if each independent patent, patent application, or publication was specifically and individually indicated to be incorporated by reference.

What is claimed is:

1. A method of diagnosing an inflammatory response in a test subject, said method comprising analyzing the level of sFlt-1 expression or activity in a sample isolated from said test subject, wherein an increased level of sFlt-1 expression or activity in said sample relative to the level found in an unaffected subject indicates that said test subject has said inflammatory response.

2. The method of claim 1, wherein said method further comprises analyzing the level of at least one of VEGF, PlGF, TNF-α, IL-6, D-dimer, E-selectin, P-selectin, ICAM-1, VCAM-1, Cox-2, or PAI-1.

3. The method of claim 1, wherein said subject is a human.

4. The method of claim 1, wherein said inflammatory response to be diagnosed is severe sepsis or septic shock.

5. A method of identifying a candidate compound useful for treating a subject with an inflammatory response, said method comprising:
(a) contacting sFlt-1 with a compound; and
(b) measuring the activity of said sFlt-1, wherein an increase in sFlt-1 activity in the presence of said compound relative to sFlt-1 activity in the absence of said compound identifies said compound as a candidate compound for treating a subject with an inflammatory response.

6. The method of claim 5, wherein said measuring step (b) comprises measuring binding of at least one of VEGF or PlGF to sFlt-1.

7. The method of claim 5, wherein said compound is selected from a chemical library.

8. A method of identifying a candidate compound useful for treating a subject with an inflammatory response, said method comprising:
(a) contacting a cell or cell extract comprising a polynucleotide encoding sFlt-1 with a compound; and
(b) measuring the level of sFlt-1 expression in said cell or cell extract, wherein an increased level of sFlt-1 expression in the presence of said compound relative to the level in the absence of said compound identifies said compound as a candidate compound for treating a subject with an inflammatory response.

9. The method of claim 8, wherein said cell is in a mammal.

10. The method of claim 9, wherein said method further comprises administering to said mammal lipopolysaccharide prior to said contacting step (a).

11. The method of claim 8, wherein said compound is selected from a chemical library.

12. A method of treating a subject with an inflammatory response, said method comprising administering to said subject a therapeutically effective amount of a composition that increases sFlt-1 expression or activity.

13. The method of claim 12, wherein said composition comprises sFlt-1.

14. The method of claim 12, wherein said method further comprises administering a treatment selected from the group consisting of antimicrobials, fluids, vasopressors, corticosteroids, activated protein C, glucose with insulin, mechanical ventilation, renal replacement therapy, and sedation.

15. The method of claim 12, wherein said subject is a human.

16. The method of claim 12, wherein said inflammatory response is severe sepsis or septic shock.

17. A method of diagnosing an inflammatory response in a test subject, said method comprising analyzing the level of PlGF expression or activity in a sample isolated from said test subject, wherein an alteration in the level of PlGF expression or activity in said sample relative to the level in an unaffected subject indicates that said test subject has said inflammatory response.

18. The method of claim 17, wherein said method further comprises analyzing the level of at least one of VEGF, PlGF, TNF-α, IL-6, D-dimer, E-selectin, P-selectin, ICAM-1, VCAM-1, Cox-2, or PAI-1.

19. The method of claim 17, wherein said subject is a human.

20. The method of claim 17, wherein said inflammatory response to be diagnosed is severe sepsis or septic shock.

21. The method of claim 17, wherein said alteration is an increase.

22. The method of claim 17, wherein said alteration is a decrease.

23. A method of identifying a candidate compound useful for treating a subject with an inflammatory response, said method comprising:
(a) contacting PlGF with a compound; and
(b) measuring the activity of said PlGF, wherein an alteration in PlGF activity in the presence of said compound relative to PlGF activity in the absence of said compound identifies said compound as a candidate compound for treating a subject with an inflammatory response.

24. The method of claim 23, wherein said compound is selected from a chemical library.

25. The method of claim 23, wherein said alteration is an increase.

26. The method of claim 23, wherein said alteration is a decrease.

27. A method of identifying a candidate compound useful for treating a subject with an inflammatory response, said method comprising:
(a) contacting a cell or cell extract comprising a polynucleotide encoding PlGF with a compound; and
(b) measuring the level of PlGF expression in said cell or cell extract, wherein an alteration in the level of PlGF expression in the presence of said compound relative to the level in the absence of said compound identifies said compound as a candidate compound for treating a subject with an inflammatory response.

28. The method of claim 27, wherein said cell is in a mammal.

29. The method of claim 28, wherein said method further comprises administering to said mammal lipopolysaccharide prior to said contacting step (a).

30. The method of claim 27, wherein said compound is selected from a chemical library.

31. The method of claim 27, wherein said alteration is an increase.

32. The method of claim 27, wherein said alteration is a decrease.

33. A method of identifying a candidate compound for treating a subject with an inflammatory response, said method comprising:
(a) contacting a PlGF receptor, or a PlGF-binding fragment thereof, with a compound; and
(b) measuring the binding of said compound to said receptor, wherein specific binding of said compound to said PlGF receptor or said fragment thereof indicates said compound is a candidate compound for treating a subject with an inflammatory response.

34. The method of claim 33, wherein said PlGF receptor is neuropilin-1 or VEGFR-1, or a fragment thereof.

35. The method of claim 33, wherein said compound is selected from a chemical library.

36. A method of treating a subject with an inflammatory response, said method comprising administering to said subject a therapeutically effective amount of a composition that alters the expression or activity of PlGF.

37. The method of claim 36, wherein said composition comprises PlGF.

38. The method of claim 36, wherein said composition comprises a nucleic acid that encodes PlGF, or a fragment thereof with PlGF activity.

39. The method of claim 36, wherein said subject is a human.

40. The method of claim 36, wherein said composition comprises an antibody specifically binds PlGF, or a PlGF-binding fragment thereof.

41. The method of claim 36, wherein said composition comprises an RNA that interferes with the mRNA coding for the PlGF protein.

42. The method of claim 36, wherein said altering is increasing.

43. The method of claim 36, wherein said altering is decreasing.

44. The method of claim 36, wherein said method further comprises administering a treatment selected from the group consisting of antimicrobials, fluids, vasopressors, corticosteroids, activated protein C, glucose with insulin, mechanical ventilation, renal replacement therapy, and sedation.

45. The method of claim 36, wherein said inflammatory response is severe sepsis or septic shock.

46. A method of treating a subject with an inflammatory response, said method comprising administering to said subject a therapeutically effective amount of a composition that alters the expression or activity of a PlGF receptor.

47. The method of claim 46, wherein said PlGF receptor is neuropilin-1 or VEGFR-1.

48. The method of claim 46, wherein said method further comprises administering a treatment selected from the group consisting of antimicrobials, fluids, vasopressors, corticosteroids, activated protein C, glucose with insulin, mechanical ventilation, renal replacement therapy, and sedation.

49. The method of claim 46, wherein said subject is a human.

50. The method of claim 46, wherein said altering is increasing.

51. The method of claim 46, wherein said altering is decreasing.

52. A method of diagnosing an inflammatory response in a test subject, said method comprising analyzing the level of VEGF expression or activity in a sample isolated from said test subject, wherein an increased level of VEGF expression or activity in said sample relative to the level found in an unaffected subject indicates that said test subject has said inflammatory response.

53. The method of claim 52, wherein said method further comprises analyzing the level of at least one of PlGF, sFlt-1, TNF-α, IL-6, D-dimer, E-selectin, P-selectin, ICAM-1, VCAM-1, Cox-2, or PAI-1.

54. The method of claim 52, wherein said subject is a human.

55. The method of claim 52, wherein said inflammatory response to be diagnosed is severe sepsis or septic shock.

56. A method of identifying a candidate compound useful for treating a subject with an inflammatory response, said method comprising:
(a) contacting VEGF with a compound; and
(b) measuring the activity of said VEGF, wherein a decrease in VEGF activity in the presence of said compound relative to VEGF activity in the absence of said compound identifies said compound as a candidate compound for treating a subject with an inflammatory response.

57. The method of claim 56, wherein said compound is selected from a chemical library.

58. A method of identifying a candidate compound useful for treating a subject with an inflammatory response, said method comprising:
(a) contacting a cell or cell extract comprising a polynucleotide encoding VEGF with a compound; and
(b) measuring the level of VEGF expression in said cell or cell extract, wherein a decreased level of VEGF expression in the presence of said compound relative to the level in the absence of said compound identifies said compound as a candidate compound for treating a subject with an inflammatory response.

59. The method of claim 58, wherein said cell is in a mammal.

60. The method of claim 59, wherein said method further comprises administering to said mammal lipopolysaccharide prior to said contacting step (a).

61. The method of claim 58, wherein said compound is selected from a chemical library.

62. A method of identifying a candidate compound for treating a subject with an inflammatory response, said method comprising:
(a) contacting a VEGF receptor, or a VEGF binding fragment thereof, with a compound; and
(b) measuring the binding of said compound to said receptor, wherein specific binding of said compound to said VEGF receptor or said fragment thereof indicates said compound is a candidate compound for treating a subject with an inflammatory response.

63. The method of claim 62, wherein said VEGF receptor is neuropilin-1, VEGFR-1, VEGFR-2, or a fragment thereof.

64. The method of claim 62, wherein said compound is selected from a chemical library.

65. A method of treating a subject with an inflammatory response, said method comprising administering to said subject a therapeutically effective amount of a composition that decreases the expression or activity of VEGF.

66. The method of claim 65, wherein said subject is a human.

67. The method of claim 65, wherein said composition comprises an antibody specifically binds VEGF, or a VEGF-binding fragment thereof.

68. The method of claim 65, wherein said composition comprises an RNA that interferes with the mRNA coding for VEGF.

69. The method of claim 65, wherein said method further comprises administering a treatment selected from the group consisting of antimicrobials, fluids, vasopressors, corticosteroids, activated protein C, glucose with insulin, mechanical ventilation, renal replacement therapy, and sedation.

70. The method of claim 65, wherein said inflammatory response is severe sepsis or septic shock.

71. A method of treating a subject with an inflammatory response, said method comprising administering to said subject a therapeutically effective amount of a composition that decreases the expression or activity of a VEGF receptor.

72. The method of claim 71, wherein said VEGF receptor is neuropilin-1, VEGFR-1, or VEGFR-2.

73. The method of claim 71, wherein said subject is a human.

74. The method of claim 71, wherein said method further comprises administering a treatment selected from the group consisting of antimicrobials, fluids, vasopressors, corticosteroids, activated protein C, glucose with insulin, mechanical ventilation, renal replacement therapy, and sedation.
